(19) United States
(12) Patent Application Publication
(10) Pub. No.: US 2008/0178309 A1
(43) Pub. Date: Jul. 24, 2008

(54) Fluorescent Indicators of Hydrogen Peroxide and Methods for Using Same

(76) Inventors: Vsevolod V. Belousov, Moscow (RU); Sergey A. Lukyanov, Moscow (RU); Arkady F. Fradkov, Moscow (RU)

Correspondence Address: PATTERSON & SHERIDAN, L.L.P., 3040 POST OAK BOULEVARD, SUITE 1500, HOUSTON, TX, 77056, US

(21) Appl. No.: 11/909,708

(22) PCT Filed: Mar. 23, 2006
(86) PCT No.: PCT/IB2006/002621

§ 371 Date: Sep. 25, 2007

Related U.S. Application Data
(60) Provisional application No. 60/665,164, filed on Mar. 25, 2005.
(60) Provisional application No. 60/772,653, filed on Feb. 13, 2006.

(51) Int. Cl. C12Q 1/68; C07K 19/00; C12N 15/11; C12N 15/63; A01K 67/027; A01H 5/00; C12N 1/21; C12N 5/10

(52) U.S. Cl. 800/13; 435/6; 530/402; 536/23.1; 435/320.1; 800/298; 435/252.8; 435/367

(57) ABSTRACT

The present invention provides a novel genetically encoded fluorescent indicator of hydrogen peroxide. The indicator comprises a sensor polypeptide which is responsive to hydrogen peroxide and a circularly permuted fluorescent protein which is operatively inserted into the sensor polypeptide. Interaction of the sensor polypeptide with hydrogen peroxide results in a change in fluorescence of the fluorescent protein. An isolated nucleic acid molecule encoding a hydrogen peroxide fluorescent indicator of the present invention is also provided. Also provided are vectors, expression cassettes, host-cells, stable cell lines, transgenic animals and transgenic plants comprising a nucleic acid of the present invention. Also provided are methods that use a fluorescent protein of the present invention or the nucleic acid encoding it.

### FIELD OF THE INVENTION

This invention relates generally to the field of biology and chemistry. More particularly, the invention is directed to fluorescent proteins.

### BACKGROUND OF THE INVENTION

In recent years there has been an increasing number of data concerning major role of oxidative stress in the development of great list of pathologies, like malignant diseases (Dreher and Junod, Eur J. Cancer. 1996, V. 32A(1), pp. 30-38; Ha et al, Clin Cancer Res. 2000, V. 6(9), pp. 3783-3787), diabetes mellitus (Baynes, Diabetes. 1991, V. 40(4), pp. 405-412), atherosclerosis (Harrison et al, Am J. Cardiol. 2003, V. 91(3A), pp. 7A-11A), chronic inflammatory processes (Araujo et al, Biofactors. 1998, V. 8(1-2), pp. 155-159), and ischemia/reperfusion injury (Sugawara and Chan, Antioxid Redox Signal. 2003, V. 5(5), pp. 597-607). The great interest is about reactive oxygen species (ROS) participation in development of neurodegenerative diseases such as trisomy 21 associated with Down's syndrome and Alzheimer disease (Busciglio and Yankner, Nature. 1995, V. 378(6559), pp. 776-779), amyotrophic lateral sclerosis (Rosen et al, Nature. 1993, V. 362(6415), pp. 59-62), Parkinson disease (Foley and Riederer, J. Neurol. 2000, V. 247, Suppl 2, pp. 1182-94) and others. The majority of pathologies driven by oxidative stress are associated with the target damage by necrosis or apoptosis of living cells and tissues (Almeida et al., Biochim Biophys Acta. 2004, V 20, pp. 59-86).

Moreover, ROS play a role in normal cell functioning and activate a number of enzymatic cascades including tyrosine kinase cascade (Yoshizumi et al, J Biol. Chem. 2000, V. 275(16), pp. 11706-11712), and MAPK-cascade (Abe et al, J Biol. Chem. 1996, V. 271(28), pp. 16586-16590) and several transcription factors such as NF-kB (Schreck et al, EMBO J. 1991, V. 10(8), pp. 2247-2258), AP-1 (Meyer et al, EMBO J. 1993, V. 12(5), pp. 2005-2015) and other molecules (Droge, Physiol Rev. 2002, V. 82(1), pp. 47-95).

A number of methods have been developed to estimate ROS production in vitro using physical methods (for example, using an electron-spin-resonance spectrometer) or chemical probes, e.g. a trans-1-(2′-Methoxyvinyl)pyrene probe was developed to measure singlet oxygen in chemical and biological systems (Posner et al, Biochem Biophys Res Commun. 1984, V. 123, pp. 869-873), and proxyl fluorescamine probes allow measurement of hydroxyl radicals and superoxide (Pou et al, Anal Biochem. 1993, V. 212, pp. 85-90). Besides chemical probes, luminescent substances, e.g. coelenterazine and lucigenin are used to measure superoxide generation (Lucas & Solano, Anal Biochem. 1992, V. 206(2), pp. 273-277; Gyllenhammar, J Immunol Methods. 1987, V. 97(2), pp. 209-213). Another approach to detect superoxide is based on the properties of chemiluminescent protein pholasin from the mollusk Pholas dactylus. However, pholasin cannot be used as an intracellular superoxide sensor because it requires an unidentified cofactor for luminescence (Muller & Campbell, J Biolumin Chemilumin. 1990, V. 5(1), pp. 25-30; Reichl et al, Free Radic Res. 2001, V. 35(6), pp. 723-733).

The main approach to detect intracellular ROS utilizes the dihydrodichlorofluorescein (DCF) derivatives (Epling et al, Cytometry. 1992, V. 13(6), pp. 615-620; Behl et al, Cell, 1994, V. 77(6), pp. 817-27; Oyama et al, Brain Res. 1993, V. 610(1), pp. 172-175). These dyes become fluorescent being loaded into a living cell and are oxidized by reactive oxygen species. This allows visualization of ROS production in the living cell in culture. However, DCF derivatives cannot be used for quantitative ROS measurement, and are sensitive to different ROS types including several reactive oxygen and nitrogen species.

Thus, there are no adequate methods to assess production of certain ROS types (e.g. singlet oxygen, superoxide anion radicals, hydrogen peroxide, hydroxyl radicals or other) in a living cell. A number of ROS measuring techniques existing at the time are either nonspecific to several types of radicals or unusable for intracellular ROS detection.

It has been shown that fluorescent proteins and derivates thereof can be used to develop genetically encoded intracellular indicators of different cellular events and conditions (WO 98/30715, Griesbeck, Curr Opin Neurobiol., 2004, v. 14(5), pp. 636-641; Bunt and Wouters, Int Rev Cytol., 2004, v. 237, pp. 205-277).

Fluorescent proteins are proteins that exhibit low, medium, or intense fluorescence upon irradiation with light of the appropriate excitation wavelength. The fluorescent characteristic of these proteins is one that arises from the interaction of two or more amino acid residues of the protein, and not from a single amino acid residue. As such, the fluorescent proteins do not include proteins that exhibit fluorescence only from residues that act by themselves as intrinsic fluors, i.e., tryptophan, tyrosine and phenylalanine. Fluorescent proteins have been isolated from the various organisms, including Cnidaria and Arthropoda species (Prasher et al., Gene 1992, V. 111(2), pp. 229-233, Matz et al., Nat. Biotechnol., 1999, V. 17(10), pp. 969-973; Xia et al., Mar Biotechnol., 2002, V. 4(2), pp. 155-62; Gurskaya et al., Biochem J., 2003, V. 373(Pt 2), pp. 403-408; Shagin et al., Mol Biol Evol., 2004, V. 21(5), pp. 841-850). A number of biological and biomedical applications of these proteins are discussed in detail by Lippincott-Schwartz and Patterson in Science, 2003, V. 300(5616), pp. 87-91.

Several types of intracellular fluorescent indicators have been developed on the basis of Aequorea victoria green fluorescent protein (GFP) and mutants thereof. For example, pH-sensitive, chlorine-anion-sensitive and redox potential-sensitive GFP variants have been generated (Kneen et al., Biophys J., 1998, V. 74(3), pp. 1591-9; Jayaraman et al., J Biol. Chem., 2000, V. 275(9), pp. 6047-50; Dooley et al., J Biol. Chem., 2004, V. 279(21), pp. 22284-93).

As used herein the term “fluorescent indicator” refers to a fluorescent protein having a sensor polypeptide whose spectral properties vary with the response state or conformation of the sensor polypeptide upon interaction with a chemical, biological, electrical or physiological parameter.

Also chimeric constructs comprising a single reporter molecule (i.e. fluorescent protein) and sensor polypeptide that are responsive to a certain parameter have been developed. For example fluorescent indicators to measure calcium concentration and membrane potential have been proposed as fluorescent indicators (Siegel et al., Neuron, 1997, V. 19(4), pp. 735-41; Nagai et al., Proc Natl Acad Sci USA, 2001, V 98(6), pp. 3197-202). In these indicators, interaction of the sensor polypeptide with the parameter results in a change of the sensor protein conformation or state that, in turn, forces fluorescent protein rearrangement and consecutive changes of fluorescent properties. However due to the very stable GFP structure, only modest changes in fluorescence of the indicators were achieved.

Use of GFP fusion proteins as partners for fluorescence resonance energy transfer (FRET) represents a more effective approach to generate fluorescent indicators. These indicators consist of two fluorescent proteins (that are able to form a FRET-pair) linked by a sensitive domain. For example, these indicators were developed to measure changes of intracellular calcium concentration (Miyawaki et al., Nature, 1997, V. 388, pp. 882-887), and detect kinase activity (Ting et al., Proc. Natl. Acad. Sci. USA, 2001, V. 98, pp. 15003-15008; Sato et al., Nature Biotechnol., 2002, V. 20, pp. 287-294).

A great deal of the research is being performed to produce circularly permuted proteins with more labile structure than basic GFP-like proteins (Baird et al., Proc. Natl. Acad. Sci. USA, 1999, v. 96, pp. 11241-11246). As used herein the term “circularly permuted fluorescent protein” means an engineered fluorescent protein comprising a linker moiety linking the amino-terminal and carboxy-terminal amino acids of an initial fluorescent protein, wherein the amino and carboxy termini are linked as internal amino acids in the circularly permuted fluorescent protein moiety; and two terminal ends, wherein the first end is an amino-terminal end and the second end is a carboxy terminal end and wherein the amino and carboxy terminal ends of the circularly permuted fluorescent protein moiety are different from the amino-terminal and carboxy-terminal amino acids of the initial fluorescent protein. Circularly permuted fluorescent proteins have been used to generate several Ca²⁺ indicators, that comprise a circularly permuted fluorescent protein (cpFP) and a sensor polypeptide inserted into cpFP molecule (Nagai et al., Proc Natl Acad Sci USA, 2001, V. 98(6), pp. 3197-3202; Nagai et al., Proc Natl Acad Sci USA, 2004, V. 101(29), pp 10554-10559). Fluorescent indicators utilizing cpFP were found the most effective because they demonstrate a lower percentage of mis-targeting and have higher signal-to-noise ratio (Filippin et al., J Biol. Chem., 2003, V. 278(40), pp. 39224-34).

### SUMMARY OF THE INVENTION

The present invention provides a novel genetically encoded fluorescent indicator of hydrogen peroxide; the indicator comprises a sensor polypeptide which is responsive to hydrogen peroxide (H₂O₂) and a circularly permuted fluorescent protein (cpFP) which is operatively inserted into the flexible region of the sensor polypeptide. Interaction of the sensor polypeptide with hydrogen peroxide results in a change in fluorescence properties of the circularly permuted fluorescent protein.

In certain embodiments, the sensor polypeptide is a pro- or eukaryotic polypeptide sensitive to hydrogen peroxide. In the preferred embodiments, the sensor polypeptide is a hydrogen peroxide-sensitive protein from a LysR family of prokaryotic transcriptional regulatory proteins (e.g. OxyR protein) or a functional fragment thereof, e.g. H₂O₂-sensitive domain thereof, for example LysR substrate binding domain. In the preferred embodiments, the sensor polypeptide is a H₂O₂-sensitive regulatory domain of OxyR protein (e.g. polypeptide comprising an amino acid sequences shown in SEQ ID NOS: 12, 14, 16, 18, 20, 22, or homologue thereof).

In certain embodiments, the circularly permuted fluorescent protein is developed from any fluorescent protein of the GFP-family, for example, from the Cnidaria or Arthropoda fluorescent protein, or a mutant thereof. The cpFPs of interest include circularly permuted Aequorea victoria GFP or mutants thereof, e.g. CFP, circularly permuted YFP, circularly permuted EGFP, EYFP, or ECFP; circularly permuted Aequorea coerulescens GFP or mutants thereof; and circularly permuted Aequorea macrodactyla GFP or mutants thereof. In preferred embodiments, the circularly permuted fluorescent protein of interest has an amino acid sequence that is homologous, substantially the same as, or identical to the amino acid sequence shown in SEQ ID NOs: 2, 4, 6, 8, or 10.

In certain embodiments, the circularly permuted fluorescent protein is operatively inserted into the flexible region of the sensor polypeptide moiety. For example, the circularly permuted fluorescent protein may be inserted into the flexible region of the H₂O₂-sensitive regulatory domain of OxyR protein, e.g. between aa 125-143 of the SEQ ID NO: 12. In certain embodiments, the N- and C-terminal amino acids of the cpFP is linked with a sensor polypeptide through short linker moieties, for example a Ser-Ala-Gly tripeptide linker at the N-terminus of the cpFP and Gly-Thr, Ser-Asp, His-Gly, or His-Asn dipeptide linker at the C-terminus of the cpFP.

A hydrogen peroxide fluorescent indicator comprising an amino acid sequence selected from the group consisting of SEQ ID NOS: 24, 26, 28, 30, 32, 34, 36, 38 or that is homologous, substantially the same as, or identical thereto is also provided.

Additionally, a fusion protein comprising a fluorescent indicator of the invention is provided. For example the indicator may have a localization sequence to target the indicator, for example, to a particular cell organelle or a cell type.

An isolated nucleic acid molecule encoding a fluorescent indicator of hydrogen peroxide of the present invention is also provided. In certain embodiments, the nucleic acid of the present invention encodes a hydrogen peroxide fluorescent indicator comprising a sensor polypeptide which is responsive to hydrogen peroxide (H₂O₂) and cpFP which is operatively inserted into the sensor polypeptide using linker moieties. A nucleic acid encoding a fusion of a hydrogen peroxide fluorescent indicator, for example with a localization sequence, is also provided.

In certain embodiments, the nucleic acid of the present invention encodes a hydrogen peroxide fluorescent indicator comprising an amino acid sequence selected from the group consisting of SEQ ID NOS: 24, 26, 28, 30, 32, 34, 36, 38 or that is homologous, substantially the same as, or identical thereto.

In certain embodiments, the nucleic acid of the present invention encodes fluorescent indicator of hydrogen peroxide and comprises a continuous or discontinuous nucleotide sequence, selected from the group consisting of SEQ ID NOs: 1, 3, 5, 7, 9, 11, 13, 15, 17, 19, 21, 23, 25, 27, 29, 31, 33, 35, 37 or that is homologous, substantially the same as, or identical thereto.

In yet other embodiments there are provided vectors comprising a nucleic acid encoding a fluorescent indicator of the present invention. In addition, the present invention provides expression cassettes comprising a nucleic acid encoding a fluorescent indicator of the present invention and regulatory elements necessary for expression of the nucleic acid in the desired host-cells.

Additionally, host-cells, stable cell lines, transgenic animals and transgenic plants comprising nucleic acids, vectors or expression cassettes of the present invention are provided.

Also provided are methods that use a fluorescent indicator of the present invention or a nucleic acid encoding it.

In preferred embodiments, the present invention provides methods for detection of hydrogen peroxide in a biological sample (e.g. biological fluid, extracellular matrix, intracellular compartment, cell cytoplasm, cell compartment(s)), wherein the methods comprise (i) contacting the sample with the fluorescent indicator of the invention and (ii) detecting a change in spectral properties of the fluorescent indicator, wherein the change in spectral properties suggests the presence of hydrogen peroxide in the sample. In the preferred embodiments, the fluorescent indicator is produced in the sample from the nucleic acid of the present invention, i.e., a nucleic acid encoding a fluorescent indicator of the present invention operatively linked with suitable regulatory elements is introduced into a sample to express subject fluorescent indicator.

Additionally, kits comprising nucleic acids or vectors or expression cassettes harboring said nucleic acids, or proteins of the present invention are provided.

### BRIEF DESCRIPTION OF THE FIGURES

FIG. 1 illustrates the excitation spectra of OxyR-RD(1-125)-Ser-Ala-Gly-cpYFP1-Gly-Thr-OxyR-RD(126-230) protein in E coli cell suspension upon addition of different concentrations of H₂O₂: line 1—no hydrogen peroxide; line 2—in the presence of 20 μM hydrogen peroxide; line 3—in the presence of 50 μM hydrogen peroxide. Emission was measured at 530 nm.

FIGS. 2A-2B illustrate the emission and excitation spectra of RI2 (FIG. 2 A), and RI7 (FIG. 2 B) proteins in E coli cell suspension. Excitation spectra were recorded at emission at 520 nm upon addition of different concentrations of H₂O₂: line 1—no hydrogen peroxide; line 2—in the presence of 20 μM hydrogen peroxide; line 3—in the presence of 50 μM hydrogen peroxide. Emission spectra (line 4) were recorded at excitation at 460 nm.

FIGS. 3A-3B illustrate spectral properties of HyPer. FIG. 3A illustrates HyPer emission (line 1) and excitation (line 2) spectra. The excitation spectrum has two maxima at 420 nm and 500 nm. Emission spectrum has a maximum at 516 nm.

FIG. 3B illustrates excitation spectrum of HyPer in Tris-HCl, pH 7.5, 150 mM NaCl, 0.5 mM 2-mercaptoethanol, upon addition of different concentrations of H₂O₂: line 1—no hydrogen peroxide; line 2—in the presence of 25 μM hydrogen peroxide; line 3—in the presence of 100 μM hydrogen peroxide; line 4—in the presence of 250 μM hydrogen peroxide. Emission was measured at 530 nm.

FIGS. 4A-4B illustrate HyPer protein properties in E coli cell suspension.

FIG. 4A illustrates HyPer excitation spectra upon addition of different concentrations of H₂O₂: line 1—no hydrogen peroxide; lines 2-5—in the presence 5 μM, 10 μM, 20 μM, 50 μM, of hydrogen peroxide, respectively. Emission was measured at 530 nm. FIG. 4B illustrates kinetics of fluorescence (excitation at 490 nm, emission at 530 nm) of HyPer in presence of catalase in response to three successive additions of H₂O₂ (arrows).

FIGS. 5A-5B illustrate the use of HyPer expressed in a cell cytoplasm to detect H₂O₂ production during apoptosis. FIG. 5A shows fluorescence intensities of HyPer-C and TMRM in a cell undergoing Apo2L/TRAIL-induced apoptosis. FIG. 5B shows fluorescence intensities of HyPer-C and TMRM in a cell incubated with ZVAD-fmk prior to the addition of TRAIL.

FIGS. 6A-6B illustrate the use of HyPer expressed in cell mitochondria to detect H₂O₂ production during apoptosis. FIG. 6A shows fluorescence intensity of HyPer-M (white columns) and TMRM (hatched columns) in a single mitochondrion at 124, 126, and 128 min. FIG. 6B shows bulk fluorescence intensity of HyPer-M and TMRM in a HeLa cell undergoing Apo2L/TRAIL-induced apoptosis.

FIG. 7 illustrates dynamics of intracellular H₂O₂ production in PC-12 cells stimulated with 100 ng/ml NGF. Lines 1 and 2—typical timecourses of HyPer fluorescence in a cell after NGF stimulation; line 3—in an untreated cell. F/Fo—ratio of fluorescence intensity level in time to fluorescence intensity at time zero.

### DETAILED DESCRIPTION OF THE INVENTION

As summarized above, the present invention is directed to a fluorescent indicator of hydrogen peroxide as well as to a nucleic acid encoding the same; the indicator comprises a sensor polypeptide which is responsive to hydrogen peroxide (H₂O₂) and a circularly permuted fluorescent protein which is operatively inserted into the sensor polypeptide using linker moieties. Interaction of the sensor polypeptide with H₂O₂ results in a change in spectral characteristics of the fluorescent protein. Also provided are vectors, expression cassettes, host-cells, stable cell lines and transgenic organisms comprising the above-referenced nucleic acid molecule. The subject fluorescent indicator and nucleic acid compositions find use in a variety of different applications and methods, particularly hydrogen peroxide measurement. Finally, kits for use in such methods and applications are provided.

### Fluorescent Indicator Composition

As summarized above the present invention provides a fluorescent indicator of hydrogen peroxide, i.e. spectral properties of the subject fluorescent indicator varies upon interaction with a hydrogen peroxide. The fluorescent indicator of the present invention comprises a sensor polypeptide which is responsive to hydrogen peroxide (H₂O₂) and a circularly permuted fluorescent protein which is operatively inserted into the sensor polypeptide. In preferred embodiments, the circularly permuted fluorescent protein is operatively inserted into the sensor polypeptide through short linker moieties.

As used herein, “operatively inserted” means between two amino acids of a polypeptide or two nucleotides of a nucleic acid sequence. Accordingly, insertion excludes ligating or attaching a polypeptide to the last terminal amino acid or nucleotide in a sequence.

In certain embodiments, the circularly permuted fluorescent protein of the present invention is developed from a fluorescent protein of the GFP family. The family includes a great deal of proteins from different sources (for example Cnidaria and Arthropoda fluorescent proteins) that share the GFP-like “beta-can” fold and are capable of autocatalytic chromophore synthesis (Shagin et al., Mol. Biol. Evol. 2004, V. 21(5), pp 841-850).

Circular permutation is usually performed at the nucleic acid level using methods known in the art, e.g. the circularly permutation technique described in U.S. Pat. Nos. 6,469,154 and 6,699,687; International Patent Application WO 00/71565 or in the Example section. Specific cpFPs of interest include circularly permuted Aequorea victoria GFP or mutants thereof, e.g. circularly permuted circularly permuted CFP, circularly permuted YFP, circularly permuted EGFP, EYFP, or ECFP; circularly permuted Aequorea coerulescens GFP or mutants thereof; and circularly permuted Aequorea macrodactyla GFP or mutants thereof. More specifically, specific proteins of interest include circularly permuted fluorescent proteins comprising amino acid sequences shown in SEQ ID NOs: 2, 4, 6, 8, 10 or homologues, substantially the same as, or identical thereto.

As used herein, “homologue or homology” is a term used in the art to describe the relatedness of a nucleotide or peptide sequence to another nucleotide or peptide sequence, which is determined by the degree of identity and/or similarity between said sequences compared.

By homolog is meant a protein or a nucleic acid having at least about 30%, usually at least about 40% and more usually at least about 60% amino acid sequence identity to referred amino acid or nucleic acid sequences. In many embodiments, homologs of interest have much higher sequence identity e.g., 65%, 70%, 75%, 80%, 85%, 90% (e.g., 92%, 93%, 94%) or higher, e.g., 95%, 96%, 97%, 98%, 99%, 99.5%, particularly for the sequence of the amino acids or nucleic acids that provide the functional regions of the protein.

By “substantially identical” is meant a protein or a nucleic acid that exhibits at least 70%, preferably 75%, more preferably 80%, and most preferably 90%, 95%, 96%, 97%, 98%, 99% or 100% sequence identity to a reference amino acid or nucleic acid sequence. For polypeptides, the length of comparison sequences will generally be at least 16 amino acids, preferably at least 20 amino acids, more preferably at least 25 amino acids, and most preferably 33 amino acids. For nucleic acids, the length of comparison sequences will generally be at least 50 nucleotides, preferably at least 60 nucleotides, more preferably at least 75 nucleotides, and most preferably 100 nucleotides.

Sequence similarity is calculated based on a reference sequence. Algorithms for sequence analysis are known in the art, such as BLAST, described in Altschul et al., J. Mol. Biol., 215, pp. 403-10 (1990), or DNAstar clustal algorithm as described in D. G. Higgins and P. M. Sharp, “Fast and Sensitive multiple Sequence Alignments on a Microcomputer,” CABIOS, 5 pp. 151-3 (1989) (using parameters ktuple 1, gap penalty 3, window 5 and diagonals saved 5).

As summarized above, the fluorescent indicator of the present invention also comprises a sensor polypeptide which is responsive to hydrogen peroxide (H₂O₂), i.e. said polypeptide changes conformation or states upon interaction with hydrogen peroxide.

As used herein, the term “responsiveness” means any response of a polypeptide to a hydrogen peroxide. A response includes small changes, for example, a shift in the orientation of an amino acid or peptide fragment of the sensor polypeptide as well as, for example, a change in the primary, secondary, or tertiary structure of a polypeptide, including for example, changes in electrical and chemical potential or conformation. As used herein, the term “conformation” means the three-dimensional arrangement of the primary, secondary and tertiary structure of a molecule including side groups in the molecule; a change in conformation occurs when the three-dimensional structure of a molecule changes.

Specific polypeptides of interest include isolated pro- or eukaryotic naturally occurring proteins or mutants or fragments thereof, which are responsive to hydrogen peroxide. As used herein, the term “fragment” means a portion of a sensor protein which can exist in at least two different states or conformations and is responsive to hydrogen peroxide. As used herein the term “mutant” refers to a protein, in which one or more amino acids are added and/or substituted and/or deleted and/or inserted at the N-terminus, and/or the C-terminus, and/or within the native amino acid sequence of the naturally occurring protein. As used herein the term “mutant” refers to a nucleic acid molecule that encodes a mutant protein. Moreover, the term “mutant” refers to any shorter or longer version of the protein or nucleic acid herein.

Mutants are usually generated on the nucleic acid level on a template nucleic acid by modifying, deleting or adding one or more nucleotides in the template sequence, or a combination thereof, to generate a variant of the template nucleic acid. The modifications, additions or deletions can be introduced by any method well-known in the art (see for example Gustin et al., Biotechniques (1993) 14: 22; Barany, Gene (1985) 37: 111-123; and Colicelli et al., Mol. Gen. Genet. (1985) 199:537-539, Sambrook et al., Molecular Cloning: A Laboratory Manual, (1989), CSH Press, pp. 15.3-15.108).

Examples of sensor polypeptides useful in the present invention include prokaryotic OxyR proteins, and H₂O₂-sensitive regulatory domains (called LysR substrate binding domains) thereof. In the preferred embodiments, the sensor polypeptide is a H₂O₂-sensitive regulatory domain of OxyR protein. OxyR proteins of interest have been described in a wide variety of bacteria, including Escherichia coli, Salmonella typhimupium, Legionella pneumophila, Xanthomonas campestris, Colwellia psychrerythraea, Vibrio fischeri, and Actinobacillus actinomycetemcomitans. Examples of the amino acid sequences of the H₂O₂-sensitive regulatory domain of OxyR proteins are shown in SEQ ID NOS: 12, 14, 16, 18, 20, 22. Also of interest LysR substrate binding domains comprising amino acid sequences of at least 70, preferably 85, more preferably 120 residues in length that have at least 25% identity, preferably at least 35% identity, more preferably at least 50% identity, and most preferably at least 60% identity with the sequences shown in SEQ ID NOS: 12, 14, 16, 18, 20, 22, e.g. LysR substrate binding domains of the LysR family transcriptional regulators from Actinobacillus succinogenes, Mannheimia succiniciproducens, Pseudomonas putida, Rhodococcus equi, Rhodobacter sphaeroides, Xanthomonas oryzae, Streptomyces coelicolor, Streptomyces avermitilis, Idiomarina baltica, Silicibacter pomeroyi, Bacillus subtilis, Vibrio vulnificus, Vibrio splendidus, Vibrio parahaemolyticus, Chloroflexus aurantiacus, Thiomicrospira crunogena, etc; hydrogen peroxide-inducible genes activators (OxyR) of Xanthomonas axonopodis, Porphyromonas gingivalis, Salinibacter rubber, Rhodopirellula baltica, Xylella fastidiosa, Haemophilus influenzae, Pasteurella multocida, etc.

In certain embodiments, the circularly permuted fluorescent protein is operatively inserted into the flexible region of a sensor polypeptide. The inventors have discovered that a cpFP inserted into the flexible region of a sensor polypeptide provides a response related to an interaction with hydrogen peroxide. In other words, the responsiveness of the sensor polypeptide to hydrogen peroxide results in a change in fluorescence of the cpFP inserted into a flexible region of the sensor polypeptide. For example cpFP can be inserted into the H₂O₂-sensitive regulatory domain of OxyR protein in a flexible region between aa 125-143 (according to SEQ ID NO: 12).

As used herein, the term “flexible region of a sensor polypeptide” means an element in a polypeptide chain that changes its conformation or state upon interaction with a chemical, biological, electrical or physiological parameter, e.g. with a hydrogen peroxide.

In certain embodiments, the fluorescent indicator of present invention includes linker moieties, linking the N- and C-terminal amino acids of the circularly permuted fluorescent protein to the sensor polypeptide. Where a linker moiety is present, the length of the linker moiety is chosen to optimize the kinetics and specificity of responsiveness of the sensor polypeptide induced by the interaction of hydrogen peroxide with the sensor polypeptide. The linker moiety should be long enough and flexible enough to allow the sensor polypeptide to freely interact and respond to a hydrogen peroxide. The linker moiety is, preferably, a peptide moiety. The preferred linker moiety is a peptide between about one and 30 amino acid residues in length, preferably between about two and 15 amino acid residues. Examples of linking moieties include Ser-Ala-Gly tripeptide or Gly-Thr, Ser-Asp, His-Gly, or His-Asn dipeptide linkers.

Additionally, specific fluorescent indicators for hydrogen peroxide are provided. Specific fluorescent indicators of interest include indicators comprising an amino acid sequence shown in SEQ ID NOs: 24, 26, 28, 30, 32, 34, 36 or 38, or that is homologous, substantially the same as, or identical thereto.

Mutants and derivates of the fluorescent indicator of the present invention are also provided, wherein said mutants and derivates change their fluorescent properties in response to hydrogen peroxide. Mutants and derivates can be generated using standard techniques of molecular biology as described above. Derivatives can be also generated using standard techniques that includes RNA-editing, chemical modifications, posttranslational and posttranscriptional modifications and the like. For instance, derivatives can be generated by processes such as altered phosphorylation, or glycosylation, or acetylation, or lipidation, or by different types of maturation cleavage and the like.

The invention also includes functional fragments of a fluorescent indicator of the present invention. As used herein, the term “functional fragment of a fluorescent indicator” refers to fragments of a fluorescent indicator that retain an ability to change spectral properties upon interaction with a hydrogen peroxide.

The fluorescent indicators or functional fragments thereof can be produced as chimeric proteins by recombinant DNA technology. Recombinant production of a fluorescent indicator involves expressing nucleic acids having sequences that encode the protein. Nucleic acids encoding fluorescent proteins can be obtained by methods known in the art.

In certain embodiments, the fluorescent indicator of the present invention contains a means for emitting light. In certain embodiments, the fluorescent indicator of the present invention is a fluorescent protein with fluorescence that can be detected by common methods (e.g., visual screening, spectrophotometry, spectrofluorometry, fluorescent microscopy, by FACS machines, etc). In many embodiments, the subject fluorescent indicator has two excitation maximum ranging from about 300 nm to 600 nm, for example the first-ranging from about 400-420 nm, and the second from about 480-510 nm, and one emission maximum ranging from about 400 nm to 700 nm, usually from about 450 nm to 650 nm and more usually from about 470 to 550 nm, e.g. from about 490 or 520 nm. In another embodiment, the subject fluorescent indicator has one excitation maximum ranging from about 300 nm to 600 nm, for example at the 500 nm, and one emission maximum ranging from about 400 nm to 700 nm, e.g. at the 510 nm.

The responsiveness of the sensor polypeptide (e.g., a change in conformation or state) that occurs in response to interaction of the sensor polypeptide with a hydrogen peroxide causes a change in spectral properties (e.g. fluorescence) of the fluorescence indicator. The spectral properties (e.g., fluorescence) of the indicator which can be altered in response to the conformational change in the sensor polypeptide include, but are not limited to, changes in the excitation or emission spectrum, quantum yield, extinction coefficient, excited life-time and degree of self-quenching, for example.

In certain embodiments, change in the spectral properties of the indicator represents a measurable difference that can be determined by estimation of the amount of any quantitative fluorescent property, e.g., the amount of fluorescence at a particular wavelength, or the integral of fluorescence over the emission spectrum. For example, a change of fluorescence intensity can be measured using a spectrophotometer at various excitation wavelengths.

Some indicators having two excitation maxima allow ratiometric measurement of hydrogen peroxide. Ratiometric measurement means a technique that involves observing the changes in the ratio of spectral properties (e.g. fluorescent intensities) at two wavelengths. Compared with measurement of the spectral properties at one wavelength, this method reduces artifacts by minimizing the influence of extraneous factors such as the changes of the indicator concentration and excitation light intensity.

Also provided are fusion proteins comprising a fluorescent indicator of the present invention, or fragments thereof, fused, for example, to a degradation sequence, a sequence of subcellular localization, a signal peptide, or any protein or polypeptide of interest. Fusion proteins may comprise for example, a fluorescent indicator of subject invention and a second polypeptide (“a fusion partner”) fused in-frame at the N-terminus and/or C-terminus of the fluorescent indicator. Fusion partners include, but are not limited to, polypeptides that can bind antibodies specific to the fusion partner (e.g., epitope tags), antibodies or binding fragments thereof, polypeptides that provide a catalytic function or induce a cellular response, ligands or receptors or mimetics thereof, and the like. Proteins comprising a fluorescent indicator of the present invention fused to a localization sequence that direct the indicator, for example, to a particular cell organelle or a cell type are of particular interest. Examples of the localization sequence include a nuclear localization sequence, an endoplasmic reticulum localization sequence, a peroxisome localization sequence, a Golgi apparatus targeting sequence, a mitochondrial localization sequence, a localized host protein and others. Localization sequences can be targeting sequences which are described, for example, in “Protein Targeting,” Chapter 35 of Stryer, L., Biochemistry (4th ed.), W. H. Freeman, 1995.

### Nucleic Acid Molecules

The present invention provides engineered nucleic acid molecules encoding fluorescent indicators of hydrogen peroxide or functional fragments thereof; subject fluorescent indicators comprise cpFP which is operatively inserted into the sensor polypeptide using short linker moieties. A nucleic acid molecule as used herein is a DNA, cDNA, RNA molecule or a molecule comprising modified nucleotides. In particular, said nucleic acid molecule is a recombinant DNA molecule having a continuous open reading frame that encodes a fluorescent indicator of the invention and is capable, under appropriate conditions, of being expressed as a fluorescent indicator.

Nucleic acid molecules of the present invention comprise nucleic acid sequences coding a cpFP operatively inserted into a nucleic acid coding a sensor polypeptide. In certain embodiment, nucleic acids of the present invention also comprise nucleic acid linkers between nucleic acid sequences coding cpFP and nucleic acid sequences coding sensor polypeptide moieties.

In preferred embodiments, nucleic acid molecules of the present invention comprise nucleic acid sequence coding a circularly permuted fluorescent protein (e.g. the sequence shown in SEQ ID NOs: 1, 3, 5, 7 or 9). The cpFP coding sequence is operatively inserted into a nucleic acid molecule coding OxyR protein or a functional fragment thereof, e.g. H₂O₂— sensitive regulatory domain of the OxyR protein. Examples of nucleic acid molecules coding H₂O₂— sensitive regulatory domain of the OxyR protein are shown in SEQ ID NOs: 11, 13, 15, 17, 19 and 21.

Specific nucleic acid molecules of interest include nucleic acid molecules encoding fluorescent indicators having an amino acid sequence selected from the group consisting of SEQ ID NOs: 24, 26, 28, 30, 32, 34, 36 and 38, e.g. nucleic acid molecules selected from the group consisting of SEQ ID NOS: 23, 25, 27, 29, 31, 33, 35 and 37.

The invention also encompasses nucleic acids that encode fluorescent indicators that are homologous, substantially similar to, identical to, or derived from the above mentioned fluorescent indicators (SEQ ID NOS: 24, 26, 28, 30, 32, 34, 36 and 38).

Nucleic acids that hybridize to the above-described nucleic acids under high stringency conditions, for example, at 50° C. or higher and 0.1×SSC (15 mM sodium chloride/1.5 mM sodium citrate) are also provided. Nucleic acids having a region of substantial identity to the provided sequences, e.g., genetically-altered versions of the nucleic acid, etc., bind to the provided sequences under high stringency hybridization conditions. By using probes, particularly labeled probes of DNA sequences, one can isolate homologous or related genes.

Also provided are nucleic acids that hybridize to the above-described nucleic acids under stringent conditions, preferably under high stringency conditions (i.e., complements of the previously-described nucleic acids). An example of stringent conditions is hybridization at 50° C. or higher and 0.1×SSC (15 mM sodium chloride/1.5 mM sodium citrate). Another example of high stringency hybridization conditions is overnight incubation at 42° C. in a solution of 50% formamide, 5×SSC, 50 mM sodium phosphate (pH7.6), 5×Denhardt's solution, 10% dextran sulfate, and 20 μg/ml denatured, sheared salmon sperm DNA, followed by washing in 0.1×SSC at about 65° C. Other high stringency hybridization conditions are known in the art and may also be used to identify nucleic acids of the invention.

Nucleic acids encoding derivates and mutants of the fluorescent indicators of the invention are also provided, wherein said variants and mutants are capable of changing fluorescent properties in response to hydrogen peroxide.

In addition, degenerate variants of the nucleic acids that encode the fluorescent indicators of the present invention are also provided. Degenerate variants of nucleic acids comprise replacements of the codons of the nucleic acid with other codons encoding the same amino acids. In particular, degenerate variants of the nucleic acids are generated to increase expression in a host cell. In this embodiment, codons of the nucleic acid that are non-preferred or a less preferred in genes in the host cell are replaced with the codons over-represented in coding sequences in genes in the host cell, wherein said replaced codons encodes the same amino acid. Humanized versions of the nucleic acids of the present invention are of particular interest. As used herein, the term “humanized” refers to changes made to the nucleic acid sequence to optimize the codons for expression of the protein in mammalian (human) cells (Yang et al., Nucleic Acids Research (1996) 24: 4592-45.93). Examples of degenerate variants of interest are described in more detail in experimental part, infra.

The subject nucleic acids are genetically engineered. The nucleic acids of the present invention can be generated synthetically by a number of different protocols known to those of skill in the art. Appropriate nucleic acid constructs are purified using standard recombinant DNA techniques as described in, for example, Sambrook et al., Molecular Cloning: A Laboratory Manual, 2^{nd} Ed., (1989) Cold Spring Harbor Press, Cold Spring Harbor, N.Y., and under regulations described in, e.g., United States Dept. of HHS, National Institute of Health (NIH) Guidelines for Recombinant DNA Research.

The nucleic acid molecules of the invention may encode all or a functional fragment of the subject proteins. Double- or single-stranded fragments may be obtained from the DNA sequence by chemically synthesizing oligonucleotides in accordance with conventional methods, by restriction enzyme digestion, by PCR amplification, etc.

Also provided are nucleic acids that encode fusion proteins comprising a protein of the present invention, or fragments thereof that are discussed in more detail above. For example, the nucleic acid molecules of the present invention can include a localization sequence to direct the indicator to a particular cell compartment(s) or a cell type. A localization sequence is operatively linked to the fluorescent indicator of the present invention. As used herein, the term “operatively linked” means that polypeptide components of a fusion protein are linked such that each maintains its function.

The term “operatively linked” or the like, when used to describe a link with expression regulatory elements means that said regulatory elements can direct the expression of the linked DNA sequence which encodes a fluorescent indicator or fluorescent indicator fusion protein.

Also provided are vectors and other nucleic acid constructs that comprise the subject nucleic acids. Suitable vectors include viral and non-viral vectors, plasmids, cosmids, phages, etc., preferably plasmids, and used for cloning, amplifying, expressing, transferring etc. of the nucleic acid sequence of the present invention in the appropriate host. The choice of appropriate vector is well known within the skill of the art, and many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo, typically by attaching regions of homology to the vector on the flanks of the desired nucleotide sequence. Regions of homology are added by ligation of oligonucleotides, or by polymerase chain reaction (PCR) using primers comprising both the region of homology and a portion of the desired nucleotide sequence.

Also provided are expression cassettes or systems used inter alia for the production of the subject fluorescent indicators, functional fragments or fusion proteins thereof or for replication of the subject nucleic acid molecules. The expression cassette may exist as an extrachromosomal element or may be integrated into the genome of the cell as a result of introduction of said expression cassette into the cell. For expression, the gene product encoded by the nucleic acid of the invention is expressed in any convenient expression system, including, for example, bacterial, yeast, insect, amphibian or mammalian systems. In the expression vector, a subject nucleic acid is operatively linked to a regulatory sequence that can include promoters, enhancers, terminators, operators, repressors and inducers. Methods for preparing expression cassettes or systems capable of expressing the desired product are known for a person skilled in the art.

Cell lines, which stably express the proteins of present invention, can be selected by the methods known in the art (e.g. the co-transfection with a selectable marker such as dhfr, gpt, neomycin, hygromycin allows the identification and isolation of the transfected cells that contain the gene integrated into a genome).

The above-described expression systems may be used in prokaryotic or eukaryotic hosts. Host-cells such as E. coli, B. subtilis, S. cerevisiae, insect cells in combination with baculovirus vectors, or cells of a higher organism such as vertebrates, e.g., COS 7 cells, HEK 293, CHO, Xenopus oocytes, etc., may be used for production of the protein.

When any of the above-referenced host cells, or other appropriate host cells or organisms are used to replicate and/or express the nucleic acids of the invention, the resulting replicated nucleic acid, expressed protein or polypeptide is within the scope of the invention as a product of the host cell or organism. The product may be recovered by an appropriate means known in the art.

### Transformants

The nucleic acids of the present invention can be used to generate transformants including transgenic organisms or site-specific gene modifications in cell lines. Transgenic cells of the subject invention include one or more nucleic acids according to the subject invention present as a transgene. For the purposes of the invention any suitable host cell may be used including prokaryotic (e.g. Escherichia coli, Streptomyces sp., Bacillus subtilis, Lactobacillus acidophilus, etc) or eukaryotic host-cells. Transgenic organism of the subject invention can be prokaryotic or a eukaryotic organism including bacteria, cyanobacteria, fungi, plants and animals, in which one or more of the cells of the organism contains heterologous nucleic acid of subject invention introduced by way of human intervention, such as by transgenic techniques well known in the art.

The isolated nucleic acid of the present invention can be introduced into the host by methods known in the art, for example infection, transfection, transformation or transconjugation. Techniques for transferring the nucleic acid molecules (i.e. DNA) into such organisms are widely known and provided in references such as Sambrook et al. (Molecular Cloning: A Laboratory Manual, 3^{nd} Ed., (2001) Cold Spring Harbor Press, Cold Spring Harbor, N.Y.).

In one embodiment, the transgenic organism can be a prokaryotic organism. Methods on the transformation of prokaryotic hosts are well documented in the art (for example see Sambrook et al. Molecular Cloning: A Laboratory Manual, 2nd edition (1989) Cold Spring Harbor Laboratory Press and Ausubel et al., Current Protocols in Molecular Biology (1995) John Wiley & Sons, Inc).

In another embodiment, the transgenic organism can be a fungus, for example yeast. Yeast is widely used as a vehicle for heterologous gene expression (for example see Goodey et al Yeast biotechnology, D R Berry et al, eds, (1987) Allen and Unwin, London, pp 401-429) and by King et al Molecular and Cell Biology of Yeasts, E F Walton and G T Yarronton, eds, Blackie, Glasgow (1989) pp 107-133). Several types of yeast vectors are available, including integrative vectors, which require recombination with the host genome for their maintenance, and autonomously replicating plasmid vectors.

Another host organism is an animal. Transgenic animals can be obtained by transgenic techniques well known in the art and provided in references such as Pinkert, Transgenic Animal Technology: a Laboratory Handbook, 2nd edition (2203) San Diego Academic Press; Gersenstein and Vintersten, Manipulating the Mouse Embryo: A Laboratory Manual, 3rd ed, (2002) Nagy A. (Ed), Cold Spring Harbor Laboratory; Blau et al., Laboratory Animal Medicine, 2nd Ed., (2002) Fox J. G., Anderson L. C., Loew F. M., Quimby F. W. (Eds), American Medical Association, American Psychological Association; Gene Targeting: A Practical Approach by Alexandra L. Joyner (Ed.) Oxford University Press; 2nd edition (2000). For example, transgenic animals can be obtained through homologous recombination, where the endogenous locus is altered. Alternatively, a nucleic acid construct is randomly integrated into the genome. Vectors for stable integration include plasmids, retroviruses and other animal viruses, YACs, and the like.

DNA constructs for homologous recombination will comprise at least a portion of a nucleic acid of the present invention, wherein the gene has the desired genetic modification(s), and includes regions of homology to the target locus. DNA constructs for random integration need not include regions of homology to mediate recombination. Conveniently, markers for positive and negative selection may be included. Methods for generating cells having targeted gene modifications through homologous recombination are known in the art. For various techniques for transfecting mammalian cells, see Keown et al., Meth. Enzymol. (1990) 185:527-537.

The transgenic animals may be any non-human animals including non-human mammals (e.g. mouse, rat), a bird or an amphibian, etc., and used in functional studies, drug screening and the like. Representative examples of the use of transgenic animals include those described infra.

Transgenic plants may be also produced. Methods of preparing transgenic plant cells and plants are described in U.S. Pat. Nos. 5,767,367; 5,750,870; 5,739,409; 5,689,049; 5,689,045; 5,674,731; 5,656,466; 5,633,155; 5,629,470; 5,595,896; 5,576,198; 5,538,879; 5,484,956; the disclosures of which are herein incorporated by reference. Methods of producing transgenic plants also are reviewed in Plant Biochemistry and Molecular Biology (eds. Lea and Leegood, John Wiley & Sons) (1993) pp. 275-295 and in Plant Biotechnology and Transgenic Plants (eds. Oksman-Caldentey and Barz), (2002) 719 p. Any suitable methods for producing plants may be used such as “gene-gun” approach or Agrobacterium-mediated transformation available for those skilled in the art.

### Methods of Use

The fluorescent indicators of the present invention (as well as other components of the subject invention described above) find use in a variety of applications for in vitro and in vivo detection and measurement of hydrogen peroxide production.

For example, fluorescent indicators of the present invention may be used in the methods for determining the presence of hydrogen peroxide in a chemical or biological sample, e.g. in biological fluid. The methods comprise contacting the sample with a fluorescent indicator of the invention, and measuring the amount of fluorescence at various excitation wavelengths in the presence and absence of a hydrogen peroxide, such that a change in the spectral characteristics is indicative of an affect of the hydrogen peroxide on the indicator.

A series of standards, with known levels of hydrogen peroxide can be used to generate a standard curve. The spectral characteristics, such as intensity of fluorescence at various excitation wavelengths, that occurs following exposure of the sample to the fluorescent indicator is measured, and the amount of the spectral property is then compared to the standard curve.

When the fluorescence indicator of the present invention has two excitation peaks e.g. with maximums ranging from about at 400-420 nm and 470-500 nm and one emission peak with maximum ranging from about 490-520 nm, and conformation change of the sensor polypeptide in response to interaction with a hydrogen peroxide causes the decrease of the one excitation peak and increase of the other, ratiometric measurements of H₂O₂ concentration are available.

In another embodiment, fluorescent indicators of the present invention may be used in the methods for determining the presence or production of a hydrogen peroxide in a cell or in a cell compartment(s), e.g. in mitochondria, a nucleus, etc. The methods comprise transfecting the cell with a nucleic acid encoding a fluorescent indicator or a suitable fluorescent indicator fusion operatively linked with a suitable regulatory elements providing expression of the indicator in the cell. The methods also comprise measuring the amount of fluorescence at various excitation wavelengths in the presence and absence of a hydrogen peroxide, such that a change in the spectral characteristics is indicative of an affect of the hydrogen peroxide on the indicator.

The methods described above allow determination of transient changes in hydrogen peroxide concentration in a sample or in a cell expressing the fluorescent indicator of the invention. In this case, the measurement of change in the spectral property of the indicator is performed over time.

For example, the cell expressing a fluorescent indicator may be co-transfected with other genes of interest in order to determine the effect of the gene product on the cell or the sensor polypeptide of the fluorescent indicator.

The methods described above can be used in screening assays to determine whether a compound (e.g. drug, a chemical or a biologic agent) alters hydrogen peroxide production in a cell or in a sample. In one embodiment, the assay is performed on a sample containing the fluorescent indicator in vitro. A sample containing a known amount of fluorescence is mixed with test compound. The amount of the hydrogen peroxide produced is then determined by measuring the change in the amount of a spectral property after contacting the sample with a test compound. In general a change in the spectral parameter by any measurable amount in the presence of the test compound as compared with the spectral parameter in the absence of the test compound indicates that the compound changes the hydrogen peroxide concentration in a sample.

In another embodiment, the ability of a compound to alter the hydrogen peroxide concentration in vivo is determined. In an in vivo assay, cells transfected with an expression vector encoding a fluorescent indicator or a suitable fluorescent indicator fusion are exposed to different amounts of the test compound, and the effect on the spectral parameter, such as fluorescence, in each cell can be determined. This provides a method for screening for compounds which affect cellular events. In a given cell type, any measurable change between spectral parameters in the presence of the test compound as compared with the spectral parameters in the absence of the test compound, indicates that the compound changes hydrogen peroxide concentration in a cell or a cell compartment(s).

The fluorescent indicators of the present invention also find use in applications involving the automated screening of arrays of cells expressing fluorescent indicators by using microscopic imaging and electronic analysis. Screening can be used for drug discovery and in the field of functional genomics where the subject indicators are used to determine cellular events following hydrogen peroxide production.

Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park Benjamin/Cummings Publishing Col, Inc. (1978), pp. 296-361.

### Kits

Also provided by the present invention are kits for use in practicing one or more of the above-described applications. Kits typically include the protein of the invention as such, or a nucleic acid encoding the same preferably with the elements for expressing the subject proteins, for example, a construct such as a vector comprising a nucleic acid encoding the subject protein.

The following example is offered by way of illustration and not by way of limitation.

### EXAMPLES

### Example 1

### Generation of the Fluorescent Indicators Using Circularly Permuted YFP Prepared on the Basis of Aequorea victoria Mutant EYFP

Regulatory domain of E. coli OxyR protein (OxyR-RD, SEQ ID NO: 12) was amplified from E. coli genomic DNA using primers Pr1 (SEQ ID NO: 41) and Pr2 (SEQ ID NO: 42). PCR product was treated by BamHI and HindIII restriction endonucleases and cloned into pQE-30 plasmid (Qiagen).

Two fragments of the EYFP coding sequence were amplified from the plasmid pEYFP-N1 (Clontech) by PCR with specific primers: (i) fragment 1: Pr3 (SEQ ID NO: 43) and Pr4 (SEQ ID NO: 44), and (ii) fragment 2: Pr5 (SEQ ID NO: 45) and Pr6 (SEQ ID NO: 46). Pr3 and Pr6 primers comprise a common part allowing complementary fragment annealing in the following elongation reaction: PCR products were mixed, annealed and elongated by PCR; the complete sequence of the resulting circularly permuted cpYFP was amplified by PCR with Pr4 and Pr5, digested using BamHI and HindIII restriction endonucleases, and cloned into pQE30 plasmid. To optimize folding and chromophore maturation of the cpYFP, several mutations were introduced into the coding sequence by site-directed mutagenesis: F46L, Q69K, V163A, S175G, H148D, and F64L. To obtain the protonated form of cpYFP chromophore, the mutation Y203F was also introduced by site-directed mutagenesis. Nucleic acid and amino acid sequences of the resulted cpYFP1 are shown in SEQ ID NOS: 1, 2.

To produce a fluorescent indicator of hydrogen peroxide, cpYFP1 coding sequence was inserted into the OxyR-RD sequence into the flexible region (between 125-143 aa according to SEQ ID NO: 12). Short amino acid linkers, Ser-Ala-Gly and Gly-Thr, were introduced between cpYFP and OxyR-RD. A collection of chimeric proteins having structure OxyR-RD (aa 1-N)-Ser-Ala-Gly-cpYFP1-Gly-Thr-OxyR-RD (aa N+1-230) was obtained wherein N is a number of amino acid residue from 125 to 143 of the SEQ ID NO: 12, and Ser-Ala-Gly and Gly-Thr are short amino acid linkers between cpYFP1 and OxyR-RD fragments. cpYFP1 DNA was amplified using primers Pr7 (SEQ ID NO: 47) and Pr8 (SEQ ID NO: 48). Fragments of OxyR-RD DNA were amplified using primers shown in the Table 1. PCR fragments comprising sequences OxyR-RD aa 1-N; cpYFP1; and OxyR-RD aa N+1-230 were mixed in equal amounts and amplified by PCR (5 cycles) using Pr1 and Pr2 after overlap extension.

Resulting chimeric proteins were digested using BamHI and HindIII restriction endonucleases, and cloned into pQE30 plasmid. To express chimeric proteins, expression constructs obtained were transformed into E. coli cells. All 11 proteins gave fluorescent signals with two excitation peaks (420 and 500 nm) and one emission peak at 510 nm. Cells expressing each chimeric protein were suspended in PBS and its fluorescence was analyzed in the presence of hydrogen peroxide using Varian Cary Eclipse spectrofluorimeter. In each case, hydrogen peroxide was added to cell suspension to a final concentration of 20 μM and changes in the emission spectrum of the cells was tested with excitation at 420 nm and at 500 nm.

Upon excitation at 490 nm, a fluorescence increase was detected in cell suspensions expressing OxyR-RD (1-125)-Ser-Ala-Gly-cpYFP1-Gly-Thr-OxyR-RD(126-230), and OxyR-RD(1-138)-Ser-Ala-Gly-cpYFP1-Gly-Thr-OxyR-RD(139-230) proteins, and a fluorescence decrease was observed in cell suspensions expressing OxyR-RD(1-131)-Ser-Ala-Gly-cpYFP1-Gly-Thr-OxyR-RD(132-230) and OxyR-RD(1-134)-Ser-Ala-Gly-cpYFP1-Gly-Thr-OxyR-RD(135-230) proteins. Additionally, a fluorescence decrease was observed in cell suspension expressing OxyR-RD(1-125)-Ser-Ala-Gly-cpYFP1-Gly-Thr-OxyR-RD(126-230) protein upon excitation at 420 nm. In control experiments, in presence of catalase (Sigma, USA) in the incubation medium no changes in fluorescence of these proteins were detected. Cell suspensions expressing other chimeric proteins did not give significant changes in fluorescent signal after addition of hydrogen peroxide.

Changes in fluorescence of clones OxyR-RD(1-125)-Ser-Ala-Gly-cpYFP1-Gly-Thr-OxyR-RD(126-230), OxyR-RD(1-138)-Ser-Ala-Gly-cpYFP1-Gly-Thr-OxyR-RD(139-230), OxyR-RD(1-131)-Ser-Ala-Gly-cpYFP1-Gly-Thr-OxyR-RD(132-230) and OxyR-RD(1-134)-Ser-Ala-Gly-cpYFP1-Gly-Thr-OxyR-RD(135-230) were reversible. Initial parameters of fluorescence were restored within 10-20 min. Addition of catalase (Sigma) to the medium after exposure to hydrogen peroxide accelerates reduction of sensor proteins. Nucleotide and amino acid sequences of these fluorescent indicators are shown in SEQ ID NOS: 23-30.

TABLE 1

OxyR-RD fragments amplification to prepare
fluorescent indicators of hydrogen peroxide

| | | |
|---|---|---|
| | Fragments of OxyR- | Primers to amplify |
| Chimeric protein structure | RD | OxyR-RD fragments |

| | | | |
|---|---|---|---|
| OxyR-RD(1-125)-cpYFP1- OxyR- | OxyR-RD(1-125) | PR1; | (SEQ ID NO: 41) |
| RD(126-230) | | Pr9 | (SEQ ID NO: 49) |
| | OxyR-RD(126-230) | PR10; | (SEQ ID NO: 50) |
| | | PR2 | (SEQ ID NO: 42) |
| OxyR-RD(1-126)-cpYFP1- OxyR- | OxyR-RD(1-126) | PR1; | (SEQ ID NO: 41) |
| RD(127-230) | | PR11 | (SEQ ID NO: 51) |
| | OxyR-RD(127-230) | PR12; | (SEQ ID NO: 52) |
| | | PR2 | (SEQ ID NO: 42) |
| OxyR-RD(1-138)-cpYFP1- OxyR- | OxyR-RD(1-131) | PR1; | (SEQ ID NO: 41) |
| RD(139-230) | | PR13 | (SEQ ID NO: 53) |
| | OxyR-RD(132-230) | PR14; | (SEQ ID NO: 54) |
| | | PR2 | (SEQ ID NO: 42) |
| OxyR-RD(1-133)-cpYFP1- OxyR- | OxyR-RD(1-133) | PR1; | (SEQ ID NO: 41) |
| RD(134-230) | | PR15 | (SEQ ID NO: 55) |
| | OxyR-RD(134-230) | PR16; | (SEQ ID NO: 56) |
| | | PR2 | (SEQ ID NO: 42) |
| OxyR-RD(1-134)-cpYFP1- OxyR- | OxyR-RD(1-134) | PR1; | (SEQ ID NO: 41) |
| RD(135-230) | | PR17 | (SEQ ID NO: 57) |
| | OxyR-RD(135-230) | PR18; | (SEQ ID NO: 58) |
| | | PR2 | (SEQ ID NO: 42) |
| OxyR-RD(1-135)-cpYFP1- OxyR- | OxyR-RD(1-135) | PR1; | (SEQ ID NO: 41) |
| RD(136-230) | | PR19 | (SEQ ID NO: 59) |
| | OxyR-RD(136-230) | PR20; | (SEQ ID NO: 60) |
| | | PR2 | (SEQ ID NO: 42) |
| OxyR-RD(1-137)-cpYFP1- OxyR- | OxyR-RD(1-137) | PR1; | (SEQ ID NO: 41) |
| RD(138-230) | | PR21 | (SEQ ID NO: 61) |
| | OxyR-RD(138-230) | PR22; | (SEQ ID NO: 62) |
| | | PR2 | (SEQ ID NO: 42) |
| OxyR-RD(1-138)-cpYFP1- OxyR- | OxyR-RD(1-138) | PR1; | (SEQ ID NO: 41) |
| RD(139-230) | | PR23 | (SEQ ID NO: 63) |
| | OxyR-RD(139-230) | PR24; | (SEQ ID NO: 64) |
| | | PR2 | (SEQ ID NO: 42) |
| OxyR-RD(1-139)-cpYFP1- OxyR- | OxyR-RD(1-139) | PR1; | (SEQ ID NO: 41) |
| RD(140-230) | | PR25 | (SEQ ID NO: 65) |
| | OxyR-RD(140-230) | PR26; | (SEQ ID NO: 66) |
| | | PR2 | (SEQ ID NO: 42) |
| OxyR-RD(1-141)-cpYFP1- OxyR- | OxyR-RD(1-141) | PR1; | (SEQ ID NO: 41) |
| RD(142-230) | | PR27 | (SEQ ID NO: 67) |
| | OxyR-RD(142-230) | PR28; | (SEQ ID NO: 68) |
| | | PR2 | (SEQ ID NO: 42) |
| OxyR-RD(1-142)-cpYFP1- OxyR- | OxyR-RD(1-142) | PR1; | (SEQ ID NO: 41) |
| RD(143-230) | | PR29 | (SEQ ID NO: 69) |
| | OxyR-RD(143-230) | PR30; | (SEQ ID NO: 70) |
| | | PR2 | (SEQ ID NO: 42) |

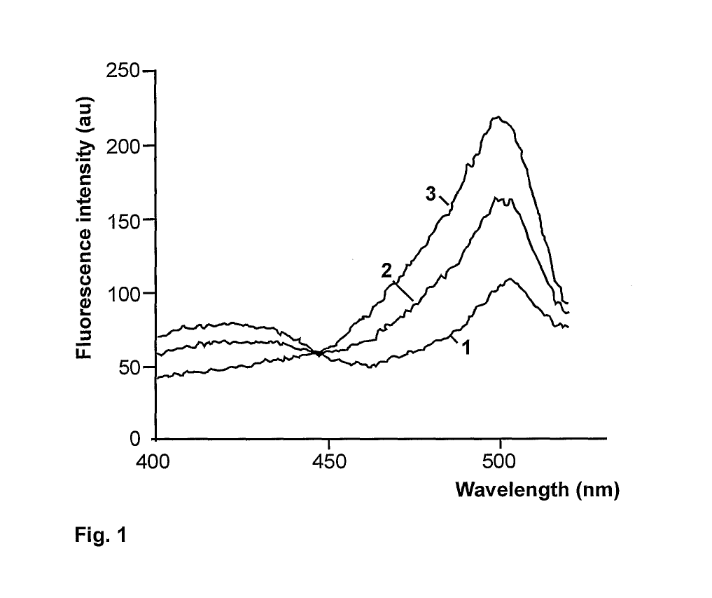

OxyR-RD(1-125)-Ser-Ala-Gly-cpYFP1-Gly-Thr-OxyR-RD(126-230) protein was selected for subsequent work as a ratiometric indicator: upon exposure to H₂O₂, the excitation peak at 420 nm decreased proportionally to the increase in the peak at 500 nm (FIG. 1).

To verify that the observed changes in fluorescence are due to OxyR-RD activity, amino acid residues corresponding to Cys199 and Cys208 of SEQ ID No:12 in the OxyR-RD were replaced by Ser. These amino acid residues have been found to crucial for OxyR-RD activity (Choi et al, Cell, 2001, V. 105(1), pp. 103-113; Lee et al, Nat Struct Mol. Biol., 2004, V. 11(12), pp. 1179-1185). All mutants in which Ser replaced one or both Cys residues showed no change in fluorescence upon exposure to H₂O₂.

To accelerate maturation speed of the indicator at 37° C., additional random mutagenesis of cpYFP moiety was performed resulting in a novel cpYFP mutant (cpYFP2) having additional substitutions I171V, G175S, A206V, and K238N. Nucleotide and amino acid sequences of the cpYFP2 are shown in SEQ ID NOs: 3, 4. Nucleotide and amino acid sequences of the fluorescent indicator comprising cpYFP2 (named HyPer) are shown in SEQ ID NOs: 31, 32.

### Example 2

### Generation of Fluorescent Indicators Using cpFP Prepared on the Basis of Aequorea macrodactyla Mutant GFP

The regulatory domain of the E. coli OxyR protein (OxyR-RD, SEQ ID NO: 12) was amplified and cloned as described in Example 1. DNA coding the Aequorea macrodactyla GFP mutant (SEQ ID NOs: 39, 40) was synthesized and cloned into pQE-30 plasmid. The mutant comprised S65C, N144S, F220L, F223S, K238R amino acid substitutions as compared with wild type Aequorea macrodactyla GFP. To prepare circularly permuted fluorescent proteins two fragments of the GFP mutant were amplified from the plasmid with specific primers: (i) fragment 1: Pr31 (SEQ ID NO: 71) and Pr32 (SEQ ID NO: 72) and (ii) fragment 2: Pr33 (SEQ ID NO: 73) and Pr34 (SEQ ID NO: 74). The primers Pr31 and Pr34 comprise sites for restriction endonucleases as well as degenerative sequences for indicator linker moieties to select that provide the best spectral characteristics of cpFPs.

PCR products were mixed, annealed and elongated by PCR; complete sequences of the resulting circularly permuted proteins were amplified by PCR with primers Pr31 and Pr34, digested using BamHI and HindIII restriction endonucleases, cloned into pQE30 plasmid and transfected into E. coli. The brightest colonies were selected and plasmid inserts were partially sequenced to find optimal linker moieties. Combinations of linker moieties selected include Ser-Ala-Gly at the N-terminal end of the cpFP and His-Gly, Ser-Asp or His-Asn at its C-terminal end. Selected cpFPs coding sequences (SEQ ID NOS: 5-10) with the linker moieties were inserted into nucleic acid molecule coding OxyR-RD (between 125 and 126 amino acid residue) as described in the Example 1. cpFPs was amplified using primers shown in the Table 2. The OxyR-RD DNA fragment for aa 1-125 was amplified using primers Pr 1 (SEQ ID NO:41) and Pr 35 (SEQ ID NO:75), and DNA fragment for aa 126-230—using primers Pr 2 (SEQ ID NO:42) and Pr 36 (SEQ ID NO:76).

TABLE 2

A. macrodactyla GFP-derived cpFP DNA amplification
to prepare fluorescent indicators of hydrogen peroxide

| | |
|---|---|
| | Primers to amplify |
| Chimeric protein structure | cpFP DNA |

| | | |
|---|---|---|
| OxyR-RD (aa 1-125) - Ser-Ala-Gly - cpFP -- His-Gly - | Pr 37; | (SEQ ID NO:77) |
| OxyR-RD (aa 126-230) | Pr 38 | (SEQ ID NO:78) |
| OxyR-RD (aa 1-125) - Ser-Ala-Gly - cpFP -- Ser-Asp - | Pr 37; | (SEQ ID NO:77) |
| OxyR-RD (aa 126-230) | Pr 39 | (SEQ ID NO:79) |
| OxyR-RD (aa 1-125) - Ser-Ala-Gly - cpFP -- His-Asn - | Pr 37; | (SEQ ID NO:77) |
| OxyR-RD (aa 126-230) | Pr 40 | (SEQ ID NO:80) |

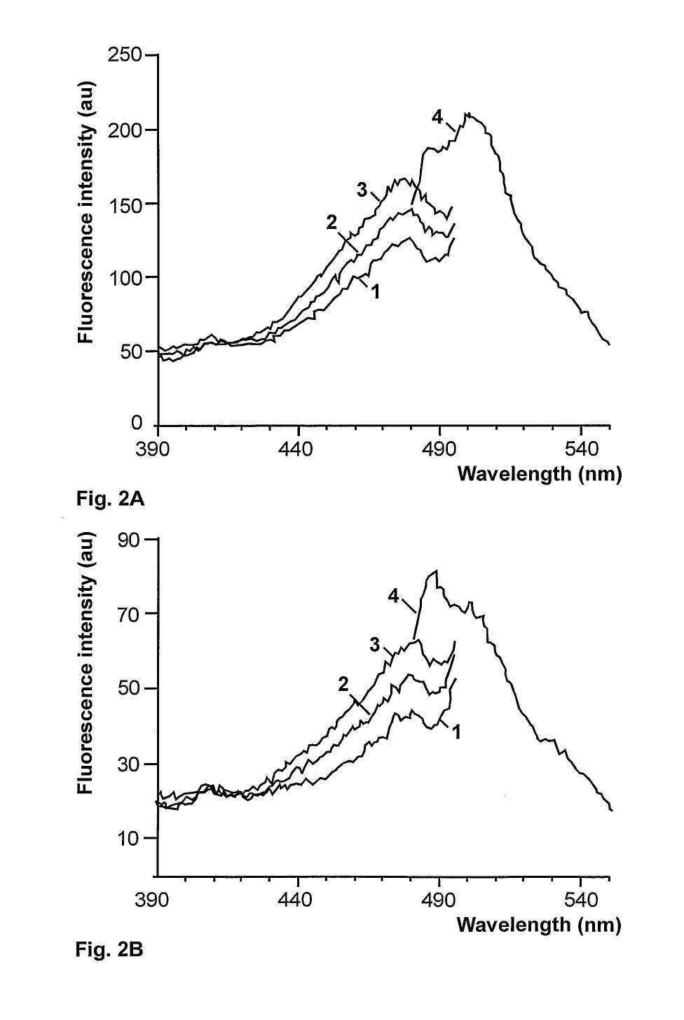

Resulting chimeric proteins were cloned into pQE30 plasmid and expressed in E. coli. The brightest colonies were selected and tested as described in the Example 1. Three nucleic acid molecules (RI2, RI6, RI7) encoding proteins with features of fluorescent indicators of hydrogen peroxide were isolated and sequenced. Nucleotide and amino acid sequences of these fluorescent indicators are shown in SEQ ID NOs: 33-38. Emission spectra and changes in excitation spectra in the presence of hydrogen peroxide for two of them are shown in FIGS. 2A-2B. The spectra were obtained on E. coli suspensions as described in the Example 1.

### Example 3

### HyPer Properties

To characterize sensitivity and selectivity of HyPer, in vitro tests using the purified protein were performed. Nucleic acid (SEQ ID NO: 31) encoding HyPer cloned into pQE-30 plasmid was prepared as described in Example 1. The protein fused to an N-terminal 6×His tag was expressed in E. coli and purified using TALON metal-affinity resin (Clontech) equilibrated with 25 mM Tris-HCl, pH 7.5, 150 mM NaCl, 5 mM 2-mercaptoethanol.

Purified protein was kept at +4° C. and aliquots of HyPer were placed into buffer containing 25 mM Tris-HCl, pH 7.5, 150 mM NaCl, 0.5 mM 2-mercaptoethanol and transferred into the spectrophotometric cuvette. After recording of initial fluorescence spectrum using a Varian Cary Eclipse spectrofluorimeter, various oxidants were added into the reaction mixture and spectrum was recorded again immediately and up to 10 min after addition.

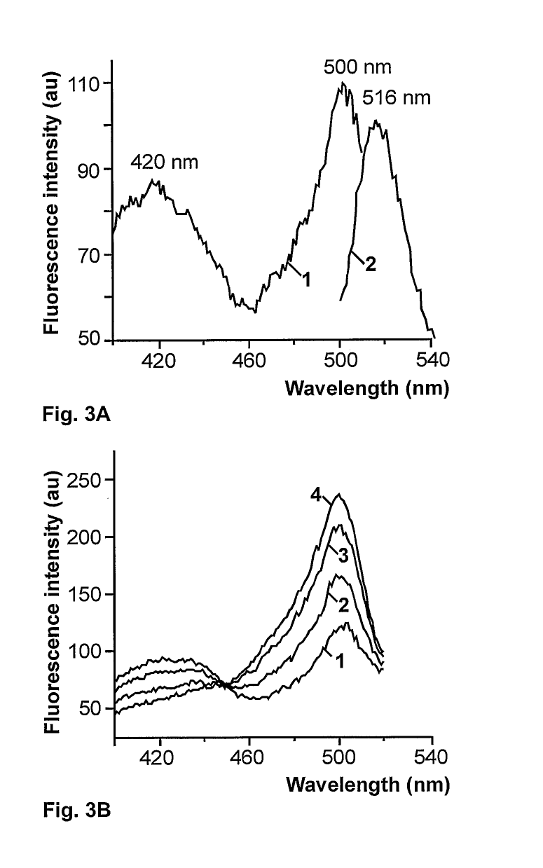

Addition of 250 nM of H₂O₂ led to complete oxidation of HyPer. The minimum concentration of H₂O₂ to induce changes in fluorescence of HyPer was 25 nM (FIGS. 3A-3B). These data show that HyPer is as sensitive as the wild type OxyR (Aslund et al., Proc Natl Acad Sci USA, 1999, v. 96, pp. 6161-6165).

To verify selectivity of HyPer, it was tested with a list of oxidants (Table 3). Except for H₂O₂ none of the compounds tested were able to induce changes in the fluorescence of HyPer.

To produce superoxide anions we used a xanthine-xanthine oxidase system (30 μM xanthine and 25 mU xanthine oxidase). To exclude possibility of HyPer oxidation by H₂O₂ generated during dismutation of superoxide, catalase was added into the reaction buffer. Indeed, in absence of catalase the xanthine-xanthine oxidase reaction led to the fast oxidation of HyPer, but addition of catalase completely prevented this process. Oxidized glutathione at 1 mM concentration was also unable to drive HyPer oxidation. Reactive nitrogen species (nitric oxide and peroxinitrite) also appeared to be unable to induce changes in HyPer fluorescence. S-nitroso-N-acetylpenicillamine (SNAP) and 3-morpholinosydnonimine (SIN-1), both in concentrations up to 0.5 mM, were used to produce nitric oxide and peroxinitrite respectively. All together, these data show that HyPer is a very sensitive and selective indicator of H₂O₂.

TABLE 3
Action of various oxidants on HyPer.

| | | Excitation ratio (500 nm/420 nm) |
|---|---|---|
| Oxidant | Source | change, folds |
| H₂O₂ | H₂O₂(25 nM) | 1.5 +/− 0.12 |
| H₂O₂ | H₂O₂(250 nM) | 3.3 +/− 0.31 |
| O2•- | Xanthine (30 μM)- | 1.0 +/− 0.04 |
| | Xanthine oxidase | |
| | (25 mU) | |
| GSSG | GSSG (1 mM) | 1.0 +/− 0.02 |
| NO | SNAP (up to 0.5 mM) | 1.0 +/− 0.05 |
| ONOO— | SIN-1 (up to 0.5 mM) | 1.0 +/− 0.04 |

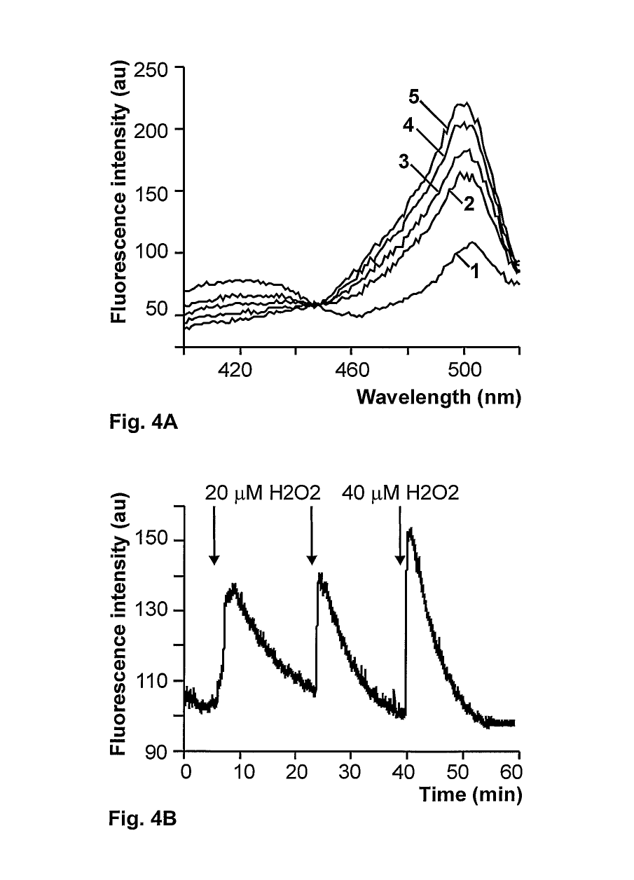

An indirect calibration of HyPer expressed in the cytoplasm of E. coli cells was also performed (FIG. 4). Cell suspension was prepared as described in Example 1, and excitation spectrum was recorded at 530 nm emission in the presence of different concentrations of hydrogen peroxide. In a suspension of the cells, the minimal concentration of added H₂O₂ needed to activate immediate changes in the fluorescence was 5 μM (FIG. 4A). The same minimal concentration of added H₂O₂ has been reported to activate wild-type OxyR in E. coli (Aslund et al, Proc Natl Acad Sci USA., 1999, V. 96(11), pp. 6161-6165). The difference in the minimal amount of H₂O₂ sufficient to oxidize HyPer in vitro and in vivo is probably due to H₂O₂ degradation by catalase and other enzymes.

Similarly to wild-type OxyR, oxidized HyPer is expected to be reduced inside cells. To test reversibility of the HyPer response we performed several cycles of oxidation of HyPer expressed in the cytoplasm of E. coli cells by repeated addition of H₂O₂ in the presence of 50 U/ml catalase (FIG. 4B). In these conditions hydrogen peroxide first reacts with HyPer and then is removed by catalase. These experiments showed that HyPer fluorescence in cells indeed restores to the initial level in several minutes after H₂O₂ burst.

Like in tests with isolated protein, no changes in HyPer fluorescence were observed in E. coli cells after addition of up to 500 μM of the nitric oxide donor S-nitroso-N-acetylpenicillamine.

To determine whether HyPer functions in mammalian cells, nucleic acid (SEQ ID NOS 9) encoding HyPer was subcloned into pEGFP-C1 vector (Clontech) in place of EGFP, under the control of CMV promoter. The vector named HyPer-C was transfected using the Lipofectamine™ 2000 (Invitrogen) into Vero and HeLa mammalian cells. The fluorescence of HyPer was detected upon irradiation of the cells with violet or blue light from a fluorescent microscope. Light microscopic imaging was performed using an Olympus CK40 fluorescent microscope. To excite the protonated form of HyPer, a violet filter D405/40x (Chroma Technology) was used, for excitation of charged form of the chromophore SZX-FGFP BP469-490 filter was used. In both cases addition of 50 μM H₂O₂ led to a fast and reversible change in the fluorescence in both channels (under blue light irradiation HyPer fluorescence decreased while green light excited fluorescence increased).

To characterize the sensitivity of HyPer in mammalian cells, a ratiometric calibration of the sensor in the cytoplasm of stably transfected COS-7 cells using fluorescence-activated cell sorting (FACS) was performed. COS-7 wild-type cells were transiently transfected with HyPer-C using the calcium phosphate method, and stable clones were selected from single cells using 1 mg/ml of G418. Cells were harvested by trypsinization and washed twice with FACS buffer (PBS containing 2% fetal bovine serum). H₂O₂ was added to the cell suspension 5 min before initiation of the flow. The 405- and 488-nm lasers of a CyAn ADP flow cytometer (DakoCytomation) were used for excitation, 530/540-nm emission filters were used for both excitation wavelengths. Data was analyzed using Summit software (DakoCytomation). 7-amino-actinomycin D was used to discriminate between live and dead cells. The minimal concentration of added H₂O₂ needed to induce changes in HyPer fluorescence was as low as 5 μM. These results showed that the OxyR-derived part of HyPer folds correctly in mammalian cells and retains its high sensitivity to H₂O₂.

### Example 4

### Visualization of H₂O₂ Production in HeLa Cells During Apoptosis

A mammalian expression vector to get HyPer to the cytosol (HyPer-C) was prepared as described in the Example 3. The HeLa cell line was transfected with the resulting constructs using the Lipofectamine™ 2000 (Invitrogen). Cells expressing HyPer-C were used to visualize changes in the H₂O₂ levels during Apo2L/TRAIL-induced apoptosis. A Leica confocal system TCS SP2 on an inverted microscope Leica DM IRE equipped with HCX PL APO lbd.BL 63×1.4NA oil objective and 125 mW Ar and 1 mW HeNe lasers was used for cell imaging. Scanning was performed using 400 Hz line frequency, 512×512 format. A green fluorescent signal was acquired using 488 nm excitation laser line (4% intensity) and detected at 500-520 nm wavelength range. Red fluorescent signal was acquired using 543 nm excitation laser line (12% intensity) and detected at 600-650 nm. Time series speed was 1 frame per 2 minutes. Quantification of image intensities was done with Leica LSC and ImageJ software (W. Rasband, National Institutes of Health, Bethesda, Md., USA).

Single-wavelength evaluation of HyPer was used to monitor H₂O₂ bursts in single living cells. For single-cell monitoring of mitochondrial transmembrane potential, HeLa cells expressing HyPer were loaded with 20 nM TMRM (Molecular Probes, Inc.) for 20 min at 37° C.

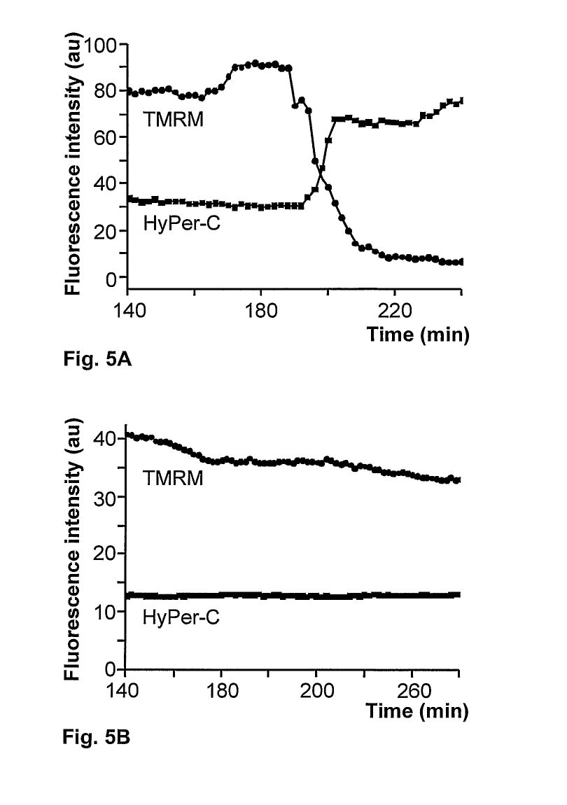

Changes in HyPer fluorescence upon excitation by 488-nm light (emission at 500-520 nm) with simultaneous visualization of TMRM fluorescence (excitation at 543 nm, emission at 600-650 nm) were examined. Because TMRM is known to produce ROS upon irradiation (Zorov et al., J Exp Med, 2000, v. 192, pp. 1001-1014), illumination conditions that would minimize TMRM-mediated production of ROS were carefully selected. Upon stimulation with Apo2L/TRAIL (400 ng/ml), HeLa cell degradation was observed. After 3-5 h, the cells changed their shape from flat to round with plasma membrane blebs. Using a combination of cytosolic-localized HyPer-C and TMRM, it was observed that cytosolic H₂O₂ started rising in parallel with a loss in the mitochondrial transmembrane potential and a change in the cell shape (FIG. 5A).

To verify that the increase in green fluorescence was not due to condensation of the cytoplasm, the ratio between the two excitation peaks of HyPer-C before and after the Apo2L/TRAIL-induced increase in green fluorescence using a Olympus CK40 fluorescent microscope was measured. It was found that Apo2L/TRAIL causes the ratio between the two peaks to change, indicating an H₂O₂-induced signal.

A loss of mitochondrial transmembrane potential occurs downstream of caspase 8 activation in Apo2L/TRAIL-induced apoptosis (Thomas et al., J. Immunol., 2000, v. 165, pp. 5612-5620). The effect of caspase inhibition on Apo2L/TRAIL-induced H₂O₂ production was investigated. Preincubation of cells with 10 μM pan-caspase inhibitor, zVAD-fmk, prevented the Apo2L/TRAIL-induced increase in H₂O₂ in the cytoplasm of the HeLa cells (FIG. 5B). This treatment also suppressed cell death and the decrease of the mitochondrial transmembrane potential. These results indicate that the increase in H₂O₂ is downstream of Apo2L/TRAIL-induced caspase activation.

To investigate the changes in the H₂O₂ level in the mitochondria of HeLa cells during Apo2L/TRAIL-induced apoptosis, cells transfected with the vector encoding mitochondrially targeted HyPer (HyPer-M) were prepared. For mitochondrial targeting, the HyPer nucleic acid was obtained as described in the Example 1 and subcloned into the pECFP-Mito vector (Clontech) in place of ECFP. To avoid mistargeting, two tandem copies of the mitochondrial targeting sequence derived from the precursor of subunit VIII of human cytochrome C oxidase were inserted into the pECFP-Mito vector.

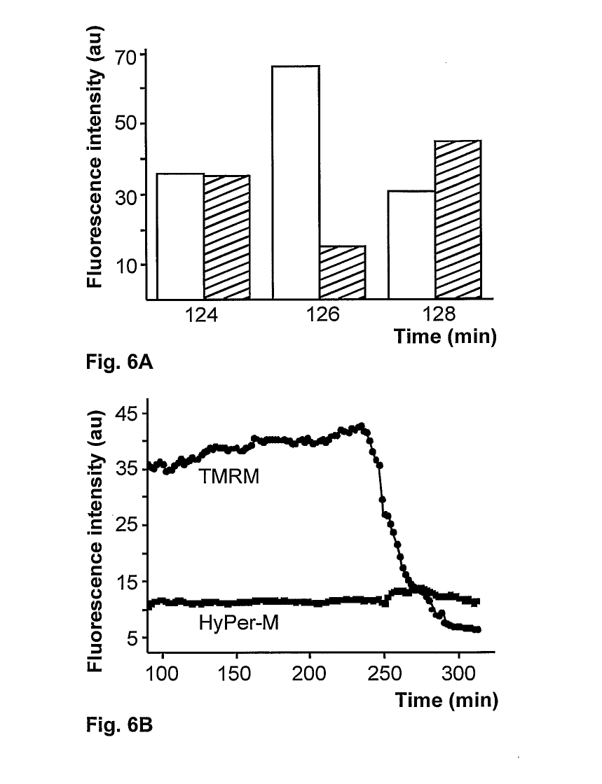

The HyPer-M cells were loaded with TMRM and then treated with Apo2L/TRAIL. After 1 to 2 hours, the transmembrane potential of some mitochondria started to oscillate; some mitochondria showed transient loss followed by restoration of the mitochondrial transmembrane potential. In depolarized mitochondria, there was an increase in the level of H₂O₂, whereas restoration of the mitochondrial transmembrane potential led to a decrease in the level of H₂O₂ (FIG. 6A). To verify that the increase in green fluorescence in depolarized mitochondria was not due to the loss of FRET between TMRM and HyPer, the cells transiently transfected with the vector encoding for mitochondria targeted CopGFP (Evrogen) were tested. Under the same conditions no changes in CopGFP fluorescence in depolarized mitochondria was observed. Without Apo2L/TRAIL, mitochondrial transmembrane potential and H₂O₂ oscillations did not occur under these conditions. These data are in agreement with previous observations that depolarized mitochondria produce ROS (Zorov et al., J Exp Med, 2000, v. 192, pp. 1001-1014). Within 1-3 hours after the start of the oscillations, all mitochondria lost their membrane potentials and the bulk mitochondrial H₂O₂ concentration rose, although not as much as in the cytoplasm (FIG. 6B).

### Example 5

### H₂O₂ Detection During Physiological Stimulation

HyPer was used to detect low-level H₂O₂, generated upon physiological stimulation (in particular by growth factors). PC-12 cells transfected with HyPer-C vector were prepared. HyPer-C vector was obtained as described in the Example 3. The cells were stimulated with the nerve growth factor (NGF), known to induce fast transient ROS production (Suzukawa et al., J Biol. Chem., 2000, v. 275, pp. 13175-13178) and changes in H₂O₂ level in the cytoplasm of stimulated cells were detected. 22 cells from 4 individual experiments were analyzed.

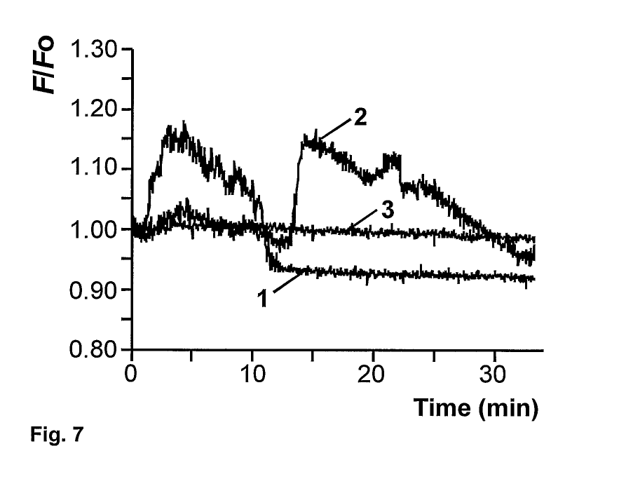

Two patterns of cellular response were observed. In most cells (n=15) H₂O₂ started to rise almost immediately after growth factor addition. The level of H₂O₂ increased and reached maximum in 3-7 min with the following decrease to the initial level by 10-20 min (FIG. 7, line 1). Also, some cells (n=7) demonstrated biphasic kinetics of hydrogen peroxide elevation. In such cells a small initial transient H₂O₂ rise was followed by a second higher and rapid increase in H₂O₂. Then HyPer fluorescence gradually reached the initial level (FIG. 7, line 2). Untreated cells demonstrated no changes in fluorescence of HyPer (FIG. 7, line 3). To ensure that these changes in fluorescence were not due to pH variations in cytoplasm of PC-12 cells after NGF treatment, the cells were incubated with the pH-sensitive dye BCECF-AM. As a result, no pH changes were detected up to 60 min after growth factor addition.

1. A genetically engineered fluorescent indicator of hydrogen peroxide, comprising a sensor polypeptide which is responsive to hydrogen peroxide and a circularly permuted fluorescent protein which is operatively inserted into a flexible region of the sensor polypeptide and wherein the spectral properties of the circularly permuted fluorescent protein are affected by the responsiveness of the sensor polypeptide to hydrogen peroxide.

2. The fluorescent indicator of claim 1 wherein the circularly permuted fluorescent protein is operatively inserted into the sensor polypeptide through short linker moieties.

3. The fluorescent indicator according to claim 1, wherein the sensor polypeptide is selected from the group consisting of:
(a) a hydrogen peroxide-sensitive protein from a LysR family of prokaryotic transcriptional regulatory proteins;
(b) a functional fragment of (a);
(c) a hydrogen peroxide-sensitive domain of (a); and
(d) LysR substrate binding domain of (a).

4. The fluorescent indicator of claim 3 wherein the sensor polypeptide is selected from the group consisting of:
(a) a polypeptide comprising an amino acid sequence as shown in SEQ ID NOs: 12, 14, 16, 18, 20, or 22;
(b) a polypeptide comprising an amino acid sequence that has at least 70% sequence identity with any contiguous or non-contiguous amino acid sequence of (a) in a region of at least 85 amino acids in length;
(c) a polypeptide comprising an amino acid sequence that has at least 50% sequence identity with any contiguous or non-contiguous amino acid sequence of (a) in a region of at least 85 amino acids in length;
(d) a polypeptide comprising an amino acid sequence that has at least 40% sequence identity with any contiguous or non-contiguous amino acid sequence of (a) in a region of at least 85 amino acids in length; and
(e) a polypeptide comprising an amino acid sequence that has at least 35% sequence identity with any contiguous or non-contiguous amino acid sequence of (a) in a region of at least 85 amino acids in length.

5. The fluorescent indicator according to claim 1, wherein the sensor polypeptide comprises an amino acid sequence as shown in SEQ ID NO: 12 or that is homologous, substantially the same as, or identical thereto.

6. The fluorescent indicator according to claim 1, wherein the circularly permuted fluorescent protein comprises an amino acid sequence that has at least 70% sequence identity with any contiguous or non-contiguous amino acid sequence shown in SEQ ID NOS: 2, 4, 6, 8, or 10 in a region of at least 100 amino acids in length.

7. The fluorescent indicator according to claim 1, wherein the fluorescent indicator comprises an amino acid sequence selected from the group consisting of:
(a) a polypeptide comprising an amino acid sequence as shown in SEQ ID NOs: 24, 26, 28, 30, 32, 34, 36, or 38;
(b) a polypeptide comprising an amino acid sequence that has at least 70% sequence identity to the amino acid sequence of at least 85 residues in length of (a);
(c) a protein having a sequence that is substantially the same as, or identical to the amino acid sequence of at least 85 residues in length of (a) or (b); and
(d) a mutant or a derivative of (a), (b), or (c).

8. A fusion protein comprising a fluorescent indicator according to claim 1.

9. The fusion protein of claim 8, wherein the fluorescent indicator is operatively fused with a specific localization signal to target the fusion protein to a desired cell compartment(s).

10. A genetically engineered nucleic acid molecule comprising a sequence encoding a fluorescent indicator according to claim 1.

11. A genetically engineered nucleic acid molecule comprising a sequence encoding a fusion protein according to claim 8.

12. A genetically engineered nucleic acid molecule comprising a sequence that encodes a fluorescent indicator of hydrogen peroxide, wherein the fluorescent indicator comprises a sensor polypeptide which is responsive to hydrogen peroxide and a circularly permuted fluorescent protein which is operatively inserted into a flexible region of the sensor polypeptide through linker polypeptide moieties, and wherein the fluorescence of the fluorescent protein is affected by the responsiveness of the sensor polypeptide to hydrogen peroxide.

13. A nucleic acid molecule of claim 12 wherein the sensor polypeptide is selected from the group consisting of:
(a) a hydrogen peroxide-sensitive protein from a LysR family of prokaryotic transcriptional regulatory proteins;
(b) a functional fragment of (a);
(c) a hydrogen peroxide-sensitive domain of (a); and
(d) a LysR substrate binding domain of (a).

14. A nucleic acid molecule of claim 12 comprising non-contiguous nucleotide sequence of the sensor polypeptide interrupted by a nucleotide sequence encoding the circularly permuted fluorescent protein and the linker polypeptide moieties, wherein the nucleotide sequence of the sensor polypeptide is selected from the group consisting of:
(a) a nucleotide sequence encoding a polypeptide that comprises an amino acid sequence as shown in SEQ ID NOs: 12, 14, 16, 18, 20, or 22;
(b) a nucleotide sequence encoding a polypeptide that has at least 70% sequence identity with any contiguous or non-contiguous amino acid sequence of (a) in a region of at least 85 amino acids in length;
(c) a nucleotide sequence encoding a polypeptide that has at least 50% sequence identity with any contiguous or non-contiguous amino acid sequence of (a) in a region of at least 85 amino acids in length;
(d) a nucleotide sequence encoding a polypeptide that has at least 40% sequence identity with any contiguous or non-contiguous amino acid sequence of (a) in a region of at least 85 amino acids in length;
(e) a nucleotide sequence encoding a polypeptide that has at least 35% sequence identity with any contiguous or non-contiguous amino acid sequence of (a) in a region of at least 85 amino acids in length; and
(f) a nucleotide sequence as shown in SEQ ID NOS: 11, 13, 15, 17, 19, or 21.

15. A nucleic acid molecule according to claim 12, wherein the circularly permuted fluorescent protein comprises an amino acid sequence having at least 70% sequence identity with any contiguous or non-contiguous amino acid sequence shown in SEQ ID NOS: 2, 4, 6, 8, or 10 in a region of at least 100 amino acids in length.

16. The nucleic acid molecule according to claim 12, selected from the group consisting of:
(a) a nucleic acid molecule encoding a fluorescent indicator that comprises the amino acid sequence as shown in SEQ ID NOs: 24, 26, 28, 30, 32, 34, 36, or 38;
(b) a nucleic acid molecule comprising a nucleotide sequence as shown in SEQ ID NOs: 23, 25, 27, 29, 31, 33, 35, or 37;
(c) a nucleic acid molecule comprising a nucleotide sequence for a fluorescent indicator that is homologous, substantially the same as, or identical to the indicator of (a) or (b);
(d) a nucleic acid molecule encoding a protein that comprises an amino acid sequence with at least 70% sequence identity to the amino acid sequence of SEQ ID NOs: 24, 26, 28, 30, 32, 34, 36, or 38 in a region of at least 85 amino acids in length;
(e) a nucleic acid molecule encoding a mutant or a derivative of (a), (b), (c) or (d); and
(f) a nucleic acid molecule that comprises a sequence encoding a functional fragment of (a), (b), (c), (d) or (e).

17. A nucleic acid molecule according to claim 10, further comprising regulatory elements for the expression of said nucleic acid molecule in desired host-cells.

18. A vector comprising a nucleic acid molecule according claim 10.

19. A cell comprising a nucleic acid molecule according claim 10.

20. A transgenic animal comprising a nucleic acid molecule according to claim 10.

21. A transgenic plant comprising a nucleic acid molecule according to claim 10.

22. A stable cell line comprising a nucleic acid molecule according to claim 10.

23. A kit comprising a nucleic acid according to claim 10.

24. A kit comprising a fluorescent indicator according to claim 1 or a means for producing the same.

25. A kit comprising a fusion protein according to claim 8 or a means for producing the same.


SEQUENCE LISTING (80 sequences)

SEQ ID NO: 1 - DNA, 738 bp, Artificial sequence
  cpYFP1 nucleotide sequence
tacaacagcgacaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaaggtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 2 - protein, 246 aa, Artificial sequence
  cpYFP1 amino acid sequence
YNSDNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYN

SEQ ID NO: 3 - DNA, 738 bp, Artificial sequence
  cpYFP2 nucleotide sequence
tacaacagcgacaacgtctatatcatggccgacaagcagaagaacggcatcaaggccaacttcaagatccgccacaacgtcgaggacggcagcgtgcagctcgccgaccactaccagcagaacacccccatcggcgacggccccgtgctgctgcccgacaaccactacctgagcttccagtccgtcctgagcaaagaccccaacgagaagcgcgatcacatggtcctgctggagttcgtgaccgccgccgggatcactctcggcatggacgagctgtacaacgtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 4 - protein, 246 aa, Artificial sequence
  cpYFP2 amino acid sequence
YNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYN

SEQ ID NO: 5 - DNA, 738 bp, Artificial sequence
  Aequorea macrodactyla mutant GFP-based cpFP1 nucleotide sequence
ttcaacagccacaacgtgtacatcatgcccgacaaggccaacaacggcctggaggcgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgagacccgcgaccacatggtgctcctggagtccttcagcgcctgcggccacacccacggcatggacgagctgtacagggtggacggcggaagcggcggaaccggcagcaagggcgaggagctgttcaccggcatcgtgcccgtgctgatcgagctggacggcgacgtgcacggccacaagttcagcgtgcgcggcgagggcgagggcgacgccgcctacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagc

SEQ ID NO: 6 - protein, 246 aa, Artificial sequence
  Aequorea macrodactyla mutant GFP-based cpFP1 amino acid sequence
FNSHNVYIMPDKANNGLEANFKIRHNIEGGGVQLADHYQTNVPLGDGPVLIPINHYLSTQTAISKDRNETRDHMVLLESFSACGHTHGMDELYRVDGGSGGTGSKGEELFTGIVPVLIELDGDVHGHKFSVRGEGEGDAAYGKLEIKFICTTGKLPVPWPTLVTTLCYGIQCFARYPEHMKMNDFFKSAMPEGYIQERTIFFQDDGKYKTRGEVKFEGDTLVNRIELKGMDFKEDGNILGHKLEYS

SEQ ID NO: 7 - DNA, 738 bp, Artificial sequence
  Aequorea macrodactyla mutant GFP-based cpFP2 nucleotide sequence
ttcaacagccacaacgtgtacatcatgcccgacaaggccaacaacggcctggaggcgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgaggcccgcgaccacatggtgctcctggagtccttcagcgcctgcggccacacccacggcatggacgagctgtacagggtggacggcggaagcggcggaaccggcagcaagggcgaggagctgttcaccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctatggcgtccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagc

SEQ ID NO: 8 - protein, 246 aa, Artificial sequence
  Aequorea macrodactyla mutant GFP-based cpFP2 amino acid sequence
FNSHNVYIMPDKANNGLEANFKIRHNIEGGGVQLADHYQTNVPLGDGPVLIPINHYLSTQTAISKDRNEARDHMVLLESFSACGHTHGMDELYRVDGGSGGTGSKGEELFTGIVPVLIELDGDVHGHKFSVRGEGEGDADYGKLEIKFICTTGKLPVPWPTLVTTLCYGVQCFARYPEHMKMNDFFKSAMPEGYIQERTIFFQDDGKYKTRGEVKFEGDTLVNRIELKGMDFKEDGNILGHKLEYS

SEQ ID NO: 9 - DNA, 738 bp, Artificial sequence
  Aequorea macrodactyla mutant GFP-based cpFP3 nucleotide sequence
ttcaacagcgacaacgtgtacatcatgcccgacaaggccaacaacggcctggaggcgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgagacccgcgaccacatggtgctcctggagtccatcagcgcctgcggccacacccacggcatggacgagctgtacagggtggacggcggaagcggcggaaccggcagcaagggcgaggagctgttcaccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagc

SEQ ID NO: 10 - protein, 249 aa, Artificial sequence
  Aequorea macrodactyla mutant GFP-based cpFP3 amino acid sequence
SAGFNSDNVYIMPDKANNGLEANFKIRHNIEGGGVQLADHYQTNVPLGDGPVLIPINHYLSTQTAISKDRNETRDHMVLLESISACGHTHGMDELYRVDGGSGGTGSKGEELFTGIVPVLIELDGDVHGHKFSVRGEGEGDADYGKLEIKFICTTGKLPVPWPTLVTTLCYGIQCFARYPEHMKMNDFFKSAMPEGYIQERTIFFQDDGKYKTRGEVKFEGDTLVNRIELKGMDFKEDGNILGHKLEYS

SEQ ID NO: 11 - DNA, 681 bp, Escherichia coli
gagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 12 - protein, 226 aa, Escherichia coli
EMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMGFCFEAGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 13 - DNA, 720 bp, Actinobacillus actinomycetemcomitans
ttattattggtggatcaggctaaaaccgtcttgcgcgaagtaaagttattaaaagaaatggcaagcaaccaaggcaaagacatgaccggtccgctacatataggcattattcctactgtaggaccttatctaatgcctcatatcgtaccgaccttacagcaaaatttccctgatctggaattattcctttatgaagcacaaacctataggttgttagaacaactggaaaccggtcgattggattgtgctattgtagcgtccgtaccggaaactgaagcctttattgaagtccaactatttaatgaaaaaatgcttcttgccgttgctgagcagcacccatgggctaatgaaaattcagtctcaatgtccttactaaaagactgtgaaatattaatgttggacgatgggcattgcttacgcaaccaagcattaggttattgcttcactgccggcgcacgggaaaacgcccactttcaggcaaccagcttagaaactctacgcaacatggtcgctgccaataccggcgtcactttaatgccacagcttgctgtgctcagtgaaggcaatcgtagtggcgtaaaatacctgccttgtgatgaaccggaaccatcccgcgacattaccttggtttatcgcccgggttcaccattacgtgcacgttatgagcgagtagcaaatacggtgagtcaatcggttaaatctatcttatcttcttaatgg

SEQ ID NO: 14 - protein, 238 aa, Actinobacillus actinomycetemcomitans
LLLVDQAKTVLREVKLLKEMASNQGKDMTGPLHIGIIPTVGPYLMPHIVPTLQQNFPDLELFLYEAQTYRLLEQLETGRLDCAIVASVPETEAFIEVQLFNEKMLLAVAEQHPWANENSVSMSLLKDCEILMLDDGHCLRNQALGYCFTAGARENAHFQATSLETLRNMVAANTGVTLMPQLAVLSEGNRSGVKYLPCDEPEPSRDITLVYRPGSPLRARYERVANTVSQSVKSILSS

SEQ ID NO: 15 - DNA, 714 bp, Vibrio fischeri ES114
gttacagttagtcgagcaagcgaaaaagatcttacttgaagtaaaacgcttttctgaacttgccaatcaacaaggtaaggaaatgacaggcccgttgcatttagggtttatacctaccgttgggccttatgtgttgccatggattgttccaacattgaaagcacagttcccagatttaaatttataccttcatgaagcgcagacgcatcaattagtgaaaatgctagaagaggggaaaattgactgtatgattttggcttctgtggaagagaccaatatgtttatcgaggttcctgtttacgatgagcctatggtacttgctgtgcctaaagatcataaatgggcaaaagaagtgtcgattgatatgtctcgtttgagtggtgaatcggttcttatgttaggagatggtcactgcttacgagatcaagccttaggtttttgttttgctgcaggggctaaagatgatgatcattttaaagcaactagtttagaaacgttacgtaatatggtcgctgccggcggggggattacattaatgccatacttgtcggtaccaaaagaaaaagagcgtgatggtgtgtgttatttacctgctcaagatcctgtccctcatcgtcaaatcgttttagcttatcgccctggttcaccactaagagcgcgatatgagtcactggctaaagagattgagaataaaatgtctaatgttattcgtag

SEQ ID NO: 16 - protein, 238 aa, Vibrio fischeri ES114
LQLVEQAKKILLEVKRFSELANQQGKEMTGPLHLGFIPTVGPYVLPWIVPTLKAQFPDLNLYLHEAQTHQLVKMLEEGKIDCMILASVEETNMFIEVPVYDEPMVLAVPKDHKWAKEVSIDMSRLSGESVLMLGDGHCLRDQALGFCFAAGAKDDDHFKATSLETLRNMVAAGGGITLMPYLSVPKEKERDGVCYLPAQDPVPHRQIVLAYRPGSPLRARYESLAKEIENKMSNVIRS

SEQ ID NO: 17 - DNA, 708 bp, Colwellia psychrerythraea 34H
acagttagttaaagaagcgcgaaaagtattacaagccgcaggacaatttgaatcttgtgctaaagccttgcttgacccgtttgcgggtgatttacatctaggattaatacctacattagcgccatatttgttgccacatatcatggcagacttgaataaagcactacctaacatcaacttctttctacatgaaaatcagaccaaggtattattgcaggaattggatgaaggtaaattagacgtattaattttgccttaccttgatgaaatggataaatttgaatcataccaattgtttgatgaaccactgatgctggcaacaccaaaaaatcatcgacttgccaataaaaaagatttatcactctccgacttacatgatgaaaaaattctaaccttagctgatggccattgtcttaaagaccaagcaatgggctattgtttttctgcgggtgctaaagaagataatagttttcaagcgacaagcttggaaacgctacgtcatatggtggcaagcggcatgggcattactttattaccagcccttgctgcacaaggaaatttagctagcgatactattcattatggtcagtttcaagccccagtgcctgtacgaggcatctctttggtgattagacctaattattcacgtatgcaatgcgttagatcgatagtggccagtgttagaaagtctctcaacggtatcattac

SEQ ID NO: 18 - protein, 236 aa, Colwellia psychrerythraea 34H
QLVKEARKVLQAAGQFESCAKALLDPFAGDLHLGLIPTLAPYLLPHIMADLNKALPNINFFLHENQTKVLLQELDEGKLDVLILPYLDEMDKFESYQLFDEPLMLATPKNHRLANKKDLSLSDLHDEKILTLADGHCLKDQAMGYCFSAGAKEDNSFQATSLETLRHMVASGMGITLLPALAAQGNLASDTIHYGQFQAPVPVRGISLVIRPNYSRMQCVRSIVASVRKSLNGIIT

SEQ ID NO: 19 - DNA, 753 bp, Xanthomonas campestris
gaagccgccgtgcgcgcgcgcagcattgtggccgaggtggaacagatgaaggaagccgcacgccgcagccaggacccggaagcgggcacggtgcgcctgggcatctttcccaccctggccccgtatttgttgccgcacgtggtaccgcgtatccgccagcgttttccgcgtctggaactgctgctgatcgaagaaaagagcgatcagctgatgcaccagctgcgcgaaggccgcctggatgccgcactgcttgcactgccattgcaggacgaccagctgcatgccgaattcctattcgaagaacccttcgtgctggcagtgcccgaaggccacccgctgtcgcgccacgacagcatgaccctggatgacctgtccgagcagcgcctgttgctgctggaagatggccactgcttgcgcgaccaggcactggatgtctgccacctggcaggcgcgctggaaaaatccgaatttcaggccaccagtctggaaaccttgcgccagatggtcgcggcgaatgtcggagtgaccttgctgccgctgttggcggtcaagccgcctgttgcgcgctcggagaacatccgcctgatccgcttccgtgaagacaagcagcctagccgccgcatcgcgatggcgtggcgccgcagttcggccatgaccgcatttctcgagcagctctcgcaactgttcaaggaattgcccgagagcctgttcacgctggatcagccggcaacgggaccgaaggcggttgcggct

SEQ ID NO: 20 - protein, 251 aa, Xanthomonas campestris
EAAVRARSIVAEVEQMKEAARRSQDPEAGTVRLGIFPTLAPYLLPHVVPRIRQRFPRLELLLIEEKSDQLMHQLREGRLDAALLALPLQDDQLHAEFLFEEPFVLAVPEGHPLSRHDSMTLDDLSEQRLLLLEDGHCLRDQALDVCHLAGALEKSEFQATSLETLRQMVAANVGVTLLPLLAVKPPVARSENIRLIRFREDKQPSRRIAMAWRRSSAMTAFLEQLSQLFKELPESLFTLDQPATGPKAVAA

SEQ ID NO: 21 - DNA, 705 bp, Legionella pneumophila str. Lens
tccaaattattagatcgaactagaaaaatattaatcctgattgatgaaatgaaagaacttgctcgtcagtcggaagatccatttacaggtgaattgcgtttgggggttatacctaccgtatcgccttacatgttgcccttggtaatgcccgaacttaaaaatgagtaccctcgcctgaaagtttggttaatagaggatcaaactcatagattaattactaaattggagcaaggagaactggatgttgctatcatggcacttccaatagataagcgtttttcatgtcaaattttatatgaagaaaagttctattttgcttgcgcaaatactcatcctttggcccaagccaaatcagttaatatcaatgacttaaaaaatcagccgattatgttgctagaagaagggcattgcctgagggagcaagccatggccgtatgtcaatcagcgaaagctgacgacatcgcagattttactgctaccagtttggaaaccttgagattaatggtacaagctggaatgggcgtcactttactgcccgctttgtctacactaactgcctcaaccaatcatttaaaatgtattcctttttctgagccagcaccctctcgtatcgtggggttattttggcgagcaggaacgccgcgacaaatttgttttaatgcaattgctgaattaataacaaaaaatgttcaatccaaattagca

SEQ ID NO: 22 - protein, 235 aa, Legionella pneumophila str. Lens
SKLLDRTRKILILIDEMKELARQSEDPFTGELRLGVIPTVSPYMLPLVMPELKNEYPRLKVWLIEDQTHRLITKLEQGELDVAIMALPIDKRFSCQILYEEKFYFACANTHPLAQAKSVNINDLKNQPIMLLEEGHCLREQAMAVCQSAKADDIADFTATSLETLRLMVQAGMGVTLLPALSTLTASTNHLKCIPFSEPAPSRIVGLFWRAGTPRQICFNAIAELITKNVQSKLA

SEQ ID NO: 23 - DNA, 1434 bp, Artificial sequence
  OxyR-RD(1-125)-Ser-Ala-Gly-cpYFP1-Gly-Thr- OxyR-RD(126-230) fluore scent indicator nucleotide sequence
gagatggcgagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggctacaacagcgacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaacgtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccacccttgtgaccaccctcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggcaccggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 24 - protein, 477 aa, Artificial sequence
  OxyR-RD(1-125)-Ser-Ala-Gly-cpYFP1-Gly-Thr- OxyR-RD(126-230) fluore scent indicator amino acid sequence
EMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMSAGYNSDNVYIMADKQKNGIKANFKIRHNIEDGSVQLADHYQQNTPIGDGPVLLPDNHYLSFQSALSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNGTGFCFEAGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 25 - DNA, 1434 bp, Artificial sequence
  OxyR-RD(1-131)-Ser-Ala-Gly-cpYFP-Gly-Thr-OxyR- RD(132-230) fluorescent indicator nucleotide sequence
gagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagcctccgccggctacaacagcgacaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaaggtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggcaccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 26 - protein, 477 aa, Artificial sequence
  OxyR-RD(1-131)-Ser-Ala-Gly-cpYFP-Gly-Thr-OxyR- RD(132-230) fluorescent indicator amino acid sequence
EMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMGFCFEASAGYNSDNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 27 - DNA, 1434 bp, Artificial sequence
  OxyR-RD(1-134)-Ser-Ala-Gly-cpYFP-Gly-Thr-OxyR- RD(135-230) fluorescent indicator nucleotide sequence
gagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggattccgccggctacaacagcgacaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaaggtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggcaccgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 28 - protein, 477 aa, Artificial sequence
  OxyR-RD(1-134)-Ser-Ala-Gly-cpYFP-Gly-Thr-OxyR- RD(135-230) fluorescent indicator amino acid sequence
EMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMGFCFEAGADSAGYNSDNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 29 - DNA, 1434 bp, Artificial sequence
  OxyR-RD(1-138)-Ser-Ala-Gly-cpYFP-Gly-Thr-OxyR- RD(139-230) fluorescent indicator nucleotide sequence
gagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggatgaagatacacactccgccggctacaacagcgacaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaaggtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggcaccttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 30 - protein, 477 aa, Artificial sequence
  OxyR-RD(1-138)-Ser-Ala-Gly-cpYFP-Gly-Thr-OxyR- RD(139-230) fluorescent indicator amino acid sequence
EMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMGFCFEAGADEDTHSAGYNSDNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGTFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 31 - DNA, 1434 bp, Artificial sequence
  HyPer nucleotide sequence
gagatggcgagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggctacaacagcgacaacgtctatatcatggccgacaagcagaagaacggcatcaaggccaacttcaagatccgccacaacgtcgaggacggcagcgtgcagctcgccgaccactaccagcagaacacccccatcggcgacggccccgtgctgctgcccgacaaccactacctgagcttccagtccgtcctgagcaaagaccccaacgagaagcgcgatcacatggtcctgctggagttcgtgaccgccgccgggatcactctcggcatggacgagctgtacaacgtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggcaccggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 32 - protein, 477 aa, Artificial sequence
  HyPer amino acid sequence
EMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMSAGYNSDNVYIMADKQKNGIKANFKIRHNVEDGSVQLADHYQQNTPIGDGPVLLPDNHYLSFQSVLSKDPNEKRDHMVLLEFVTAAGITLGMDELYNVDGGSGGTGSKGEELFTGVVPILVELDGDVNGHKFSVSGEGEGDATYGKLTLKLICTTGKLPVPWPTLVTTLGYGLKCFARYPDHMKQHDFFKSAMPEGYVQERTIFFKDDGNYKTRAEVKFEGDTLVNRIELKGIDFKEDGNILGHKLEYNGTGFCFEAGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 33 - DNA, 1437 bp, Artificial sequence
  RI2 fluorescent indicator nucleotide sequence
atggagatggcgagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggcttcaacagccacaacgtgtacatcatgcccgacaaggccaacaacggcctggaggcgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgagacccgcgaccacatggtgctcctggagtccttcagcgcctgcggccacacccacggcatggacgagctgtacagggtggacggcggaagcggcggaaccggcagcaagggcgaggagctgttcaccggcatcgtgcccgtgctgatcgagctggacggcgacgtgcacggccacaagttcagcgtgcgcggcgagggcgagggcgacgccgcctacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagccacggcggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 34 - protein, 478 aa, Artificial sequence
  RI2 fluorescent indicator amino acid sequence
MEMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMSAGFNSHNVYIMPDKANNGLEANFKIRHNIEGGGVQLADHYQTNVPLGDGPVLIPINHYLSTQTAISKDRNETRDHMVLLESFSACGHTHGMDELYRVDGGSGGTGSKGEELFTGIVPVLIELDGDVHGHKFSVRGEGEGDAAYGKLEIKFICTTGKLPVPWPTLVTTLCYGIQCFARYPEHMKMNDFFKSAMPEGYIQERTIFFQDDGKYKTRGEVKFEGDTLVNRIELKGMDFKEDGNILGHKLEYSHGGFCFEAGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 35 - DNA, 1437 bp, Artificial sequence
  RI6 fluorescent indicator nucleotide sequence
atggagatggcgagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggcttcaacagccacaacgtgtacatcatgcccgacaaggccaacaacggcctggaggcgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgaggcccgcgaccacatggtgctcctggagtccttcagcgcctgcggccacacccacggcatggacgagctgtacagggtggacggcggaagcggcggaaccggcagcaagggcgaggagctgttcaccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctatggcgtccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagctctgatggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 36 - protein, 478 aa, Artificial sequence
  RI6 fluorescent indicator amino acid sequence
MEMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMSAGFNSHNVYIMPDKANNGLEANFKIRHNIEGGGVQLADHYQTNVPLGDGPVLIPINHYLSTQTAISKDRNEARDHMVLLESFSACGHTHGMDELYRVDGGSGGTGSKGEELFTGIVPVLIELDGDVHGHKFSVRGEGEGDADYGKLEIKFICTTGKLPVPWPTLVTTLCYGVQCFARYPEHMKMNDFFKSAMPEGYIQERTIFFQDDGKYKTRGEVKFEGDTLVNRIELKGMDFKEDGNILGHKLEYSSDGFCFEAGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 37 - DNA, 1437 bp, Artificial sequence
  RI7 fluorescent indicator nucleotide sequence
atggagatggcgagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggcttcaacagcgacaacgtgtacatcatgcccgacaaggccaacaacggcctggaggcgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgagacccgcgaccacatggtgctcctggagtccatcagcgcctgcggccacacccacggcatggacgagctgtacagggtggacggcggaagcggcggaaccggcagcaagggcgaggagctgttcaccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagccataatggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 38 - protein, 478 aa, Artificial sequence
  RI7 fluorescent indicator amino acid sequence
MEMASQQGETMSGPLHIGLIPTVGPYLLPHIIPMLHQTFPKLEMYLHEAQTHQLLAQLDSGKLDCVILALVKESEAFIEVPLFDEPMLLAIYEDHPWANRECVPMADLAGEKLLMLEDGHCLRDQAMSAGFNSDNVYIMPDKANNGLEANFKIRHNIEGGGVQLADHYQTNVPLGDGPVLIPINHYLSTQTAISKDRNETRDHMVLLESISACGHTHGMDELYRVDGGSGGTGSKGEELFTGIVPVLIELDGDVHGHKFSVRGEGEGDADYGKLEIKFICTTGKLPVPWPTLVTTLCYGIQCFARYPEHMKMNDFFKSAMPEGYIQERTIFFQDDGKYKTRGEVKFEGDTLVNRIELKGMDFKEDGNILGHKLEYSHNGFCFEAGADEDTHFRATSLETLRNMVAAGSGITLLPALAVPPERKRDGVVYLPCIKPEPRRTIGLVYRPGSPLRSRYEQLAEAIRARMDGHFDKVLKQAV

SEQ ID NO: 39 - DNA, 717 bp, Artificial sequence
  Aequorea macrodactyla mutant GFP nucleotide sequence
atgagcaagggcgaggagctgttcaccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccttctgctacggcatccagtgcttcgcccgctaccccgagcacatgaagatgaacgacttcttcaagagcgccatgcccgagggctacatccaggagcgcaccatcttcttccaggacgacggcaagtacaagacccgcggcgaggtgaagttcgagggcgacaccctggtgaaccgcatcgagctgaagggcatggacttcaaggaggacggcaacatcctgggccacaagctggagtacagcttcaacagccacaacgtgtacatcatgcccgacaaggccaacaacggcctgaaggtgaacttcaagatccgccacaacatcgagggcggcggcgtgcagctggccgaccactaccagaccaacgtgcccctgggcgacggccccgtgctgatccccatcaaccactacctgagcacccagaccgccatcagcaaggaccgcaacgagacccgcgaccacatggtgctcctggagtccttcagcgcctgcggccacacccacggcatggacgagctgtacaggtga

SEQ ID NO: 40 - protein, 238 aa, Artificial sequence
  Aequorea macrodactyla mutant GFP amino acid sequence
MSKGEELFTGIVPVLIELDGDVHGHKFSVRGEGEGDADYGKLEIKFICTTGKLPVPWPTLVTTFCYGIQCFARYPEHMKMNDFFKSAMPEGYIQERTIFFQDDGKYKTRGEVKFEGDTLVNRIELKGMDFKEDGNILGHKLEYSFNSHNVYIMPDKANNGLKVNFKIRHNIEGGGVQLADHYQTNVPLGDGPVLIPINHYLSTQTAISKDRNETRDHMVLLESFSACGHTHGMDELYR

SEQ ID NO: 41 - DNA, 25 bp, Artificial sequence
  oligonucleotide primer Pr1
tattggatccgagatggcaagccag

SEQ ID NO: 42 - DNA, 24 bp, Artificial sequence
  oligonucleotide primer Pr2
ttcaaagcttttaaaccgcctgtt

SEQ ID NO: 43 - DNA, 41 bp, Artificial sequence
  oligonucleotide primer Pr3
gtggatggcggtagcggtggcaccggcagcaagggcgagga

SEQ ID NO: 44 - DNA, 34 bp, Artificial sequence
  oligonucleotide primer Pr4
atcaaagctttcaggtgccgttgtactccagctt

SEQ ID NO: 45 - DNA, 36 bp, Artificial sequence
  oligonucleotide primer Pr5
tattggatccagcgctggctacaacagcgacaacgt

SEQ ID NO: 46 - DNA, 41 bp, Artificial sequence
  oligonucleotide primer Pr6
gccggtgccaccgctaccgccatccaccttgtacagctcgt

SEQ ID NO: 47 - DNA, 24 bp, Artificial sequence
  oligonucleotide primer Pr7
tccgccggctacaacagcgacaac

SEQ ID NO: 48 - DNA, 23 bp, Artificial sequence
  oligonucleotide primer Pr8
ggtgccgttgtactccagcttgt

SEQ ID NO: 49 - DNA, 35 bp, Artificial sequence
  oligonucleotide primer Pr9
gctgttgtagccggcggacattgcctgatcgcgca

SEQ ID NO: 50 - DNA, 33 bp, Artificial sequence
  oligonucleotide primer Pr10
tggagtacaacggcaccggtttctgttttgaag

SEQ ID NO: 51 - DNA, 35 bp, Artificial sequence
  oligonucleotide primer Pr11
gctgttgtagccggcggaacccattgcctgatcgc

SEQ ID NO: 52 - DNA, 32 bp, Artificial sequence
  oligonucleotide primer Pr12
tggagtacaacggcaccttctgttttgaagcc

SEQ ID NO: 53 - DNA, 34 bp, Artificial sequence
  oligonucleotide primer Pr13
gctgttgtagccggcggaggcttcaaaacagaaa

SEQ ID NO: 54 - DNA, 33 bp, Artificial sequence
  oligonucleotide primer Pr14
tggagtacaacggcaccggggcggatgaagata

SEQ ID NO: 55 - DNA, 34 bp, Artificial sequence
  oligonucleotide primer Pr15
gctgttgtagccggcggacgccccggcttcaaaa

SEQ ID NO: 56 - DNA, 34 bp, Artificial sequence
  oligonucleotide primer Pr16
tggagtacaacggcaccgatgaagatacacactt

SEQ ID NO: 57 - DNA, 35 bp, Artificial sequence
  oligonucleotide primer Pr17
gctgttgtagccggcggaatccgccccggcttcaa

SEQ ID NO: 58 - DNA, 33 bp, Artificial sequence
  oligonucleotide primer Pr18
tggagtacaacggcaccgaagatacacacttcc

SEQ ID NO: 59 - DNA, 36 bp, Artificial sequence
  oligonucleotide primer Pr19
gctgttgtagccggcggattcatccgccccggcttc

SEQ ID NO: 60 - DNA, 32 bp, Artificial sequence
  oligonucleotide primer Pr20
tggagtacaacggcaccgatacacacttccgc

SEQ ID NO: 61 - DNA, 35 bp, Artificial sequence
  oligonucleotide primer Pr21
gctgttgtagccggcggatgtatcttcatccgccc

SEQ ID NO: 62 - DNA, 34 bp, Artificial sequence
  oligonucleotide primer Pr22
tggagtacaacggcacccacttccgcgcgaccag

SEQ ID NO: 63 - DNA, 38 bp, Artificial sequence
  oligonucleotide primer Pr23
gctgttgtagccggcggagtgtgtatcttcatccgccc

SEQ ID NO: 64 - DNA, 34 bp, Artificial sequence
  oligonucleotide primer Pr24
tggagtacaacggcaccttccgcgcgaccagcct

SEQ ID NO: 65 - DNA, 35 bp, Artificial sequence
  oligonucleotide primer Pr25
gctgttgtagccggcggagaagtgtgtatcttcat

SEQ ID NO: 66 - DNA, 35 bp, Artificial sequence
  oligonucleotide primer Pr26
tggagtacaacggcacccgcgcgaccagcctggaa

SEQ ID NO: 67 - DNA, 35 bp, Artificial sequence
  oligonucleotide primer Pr27
gctgttgtagccggcggacgcgcggaagtgtgtat

SEQ ID NO: 68 - DNA, 34 bp, Artificial sequence
  oligonucleotide primer Pr28
tggagtacaacggcaccaccagcctggaaactct

SEQ ID NO: 69 - DNA, 35 bp, Artificial sequence
  oligonucleotide primer Pr29
gctgttgtagccggcggaggtcgcgcggaagtgtg

SEQ ID NO: 70 - DNA, 32 bp, Artificial sequence
  oligonucleotide primer Pr30
tggagtacaacggcaccagcctggaaactctg

SEQ ID NO: 71 - DNA, 47 bp, Artificial sequence
  oligonucleotide primer Pr31
acatggatccgccggcttcaacagcsacaacgtgtacatcatgcccg

SEQ ID NO: 72 - DNA, 44 bp, Artificial sequence
  oligonucleotide primer Pr32
gttccgccgcttccgccgtccaccctgtacagctcgtccatgcc

SEQ ID NO: 73 - DNA, 44 bp, Artificial sequence
  oligonucleotide primer Pr33
acggcggaagcggcggaaccggcagcaagggcgaggagctgttc

SEQ ID NO: 74 - DNA, 40 bp, Artificial sequence
  oligonucleotide primer Pr34
aggtaagcttannnnnngctgtactccagcttgtggccca

SEQ ID NO: 75 - DNA, 38 bp, Artificial sequence
  oligonucleotide primer Pr35
gctgttgaagccggcggacattgcctgatcgcgcaaac

SEQ ID NO: 76 - DNA, 21 bp, Artificial sequence
  oligonucleotide primer Pr36
ggtttctgttttgaagccgga

SEQ ID NO: 77 - DNA, 21 bp, Artificial sequence
  oligonucleotide primer Pr37
atgtccgccggcttcaacagc

SEQ ID NO: 78 - DNA, 36 bp, Artificial sequence
  oligonucleotide primer Pr38
tccggcttcaaaacagaaaccgccgtcgctgtactc

SEQ ID NO: 79 - DNA, 36 bp, Artificial sequence
  oligonucleotide primer Pr39
tccggcttcaaaacagaaaccatcagagctgtactc

SEQ ID NO: 80 - DNA, 36 bp, Artificial sequence
  oligonucleotide primer Pr40
tccggcttcaaaacagaaaccattatggctgtactc